(19) United States
(12) Patent Application Publication
(10) Pub. No.: US 2024/0148760 A1
(43) Pub. Date: May 9, 2024

(54) METHODS AND COMPOSITIONS FOR CANNABINOID-BASED THERAPEUTICS

(71) Applicant: Ojai Energetics PBC, Ojai, CA (US)

(72) Inventor: William KLEIDON, Ojai, CA (US)

(21) Appl. No.: 18/202,567

(22) Filed: May 26, 2023

Related U.S. Application Data
(63) Continuation of application No. PCT/US2021/061206, filed on Nov. 30, 2021.
(60) Provisional application No. 63/119,842, filed on Dec. 1, 2020.
(60) Provisional application No. 63/119,862, filed on Dec. 1, 2020.

(51) Int. Cl. A61K 31/00; A61K 9/48; A61K 9/50; A61K 31/135; A61K 31/337; A61K 31/5513; A61K 31/704

(52) U.S. Cl. CPC A61K 31/658; A61K 9/4833; A61K 9/50; A61K 31/135; A61K 31/337; A61K 31/5513; A61K 31/704

(57) ABSTRACT

The present disclosure provides methods and systems for forming a co-therapeutic composition. The co-therapeutic composition can comprise a cannabinoid compound and a non-cannabinoid therapeutic compound. The co-therapeutic composition can comprise a plurality of stable droplets as part of an emulsion. The co-therapeutic composition can be in other dosage forms, such as solid dosage forms (e.g., tablets).

### CROSS-REFERENCE

This application is a continuation of International Patent Application No. PCT/US2021/061206, filed on Nov. 30, 2021, which claims priority to U.S. Provisional Patent Application No. 63/119,842 filed Dec. 1, 2020, and U.S. Provisional Patent Application No. 63/119,862, filed Dec. 1, 2020, each of which is entirely incorporated herein by reference.

### BACKGROUND

Therapeutic compositions suffer from drawbacks limiting their utility for administration to subjects. Many compositions are destroyed, broken down, or cleared by the liver, resulting in reduced delivery to the subject. Many therapeutic compositions also have low bioavailability and shelf life stability.

### SUMMARY

There remains a substantial need for compositions having increased bioavailability, increased shelf stability, reduced first pass metabolism, and other beneficial properties. Provided are methods for incorporating therapeutic compounds into compositions to increase bioavailability, increase shelf stability, reduce first pass metabolism, extend or modify release profiles, or increase solubility in water, such as to ease delivery and/or administration thereof to a subject. Therapeutic compositions of the present disclosure can be used to treat various diseases or conditions in subjects. In some embodiments, microencapsulation may involve generating a plurality of droplets in an emulsion.

In an aspect, the present disclosure provides a composition comprising a cannabinoid compound and a non-cannabinoid therapeutic compound, wherein the non-cannabinoid therapeutic compound is selected from the group consisting of Kyprolis, Epogen, Nplate, Vectibix, Blincyto, Corlanor, Evenity, Imlygic, Kanjinti, MVASI, Neupogen, Prolia, Repatha, Depakote, Gengraf, Kaletra, Leuplin, Mavyret, Oriahnn, Rinvoq, Survanta, Synthroid, ultane, venclexta, viekira, zemplar, Triumeq, Keytruda, Gardasil, Pneumovax, Ocrevus, Cyramza, doxorubicin, nano-inducing liposomal doxorubicin, a nano-inducing liposomal taxane, ketamine, a statin, dextromethorphan, a non-steroidal anti-inflammatory drug, a benzodiazepine, and Tables 1-10.

In an embodiment, at least one of i) the cannabinoid compound and ii) the non-cannabinoid therapeutic compound is encapsulated.

In an embodiment, the non-cannabinoid therapeutic compound is doxorubicin.

In an embodiment, the non-cannabinoid therapeutic compound is nano-inducing liposomal doxorubicin.

In an embodiment, the non-cannabinoid therapeutic compound is the nano-inducing liposomal taxane.

In an embodiment, the non-cannabinoid therapeutic compound is the ketamine.

In an embodiment, the non-cannabinoid therapeutic compound is the statin.

In an embodiment, the statin is atorvastatin.

In an embodiment, the statin is fluvastatin.

In an embodiment, the statin is lovastatin.

In an embodiment, the non-cannabinoid therapeutic compound is the dextromethorphan.

In an embodiment, the non-cannabinoid therapeutic compound is the non-steroidal anti-inflammatory drug.

In an embodiment, the non-steroidal anti-inflammatory drug is ibuprofen.

In an embodiment, the non-steroidal anti-inflammatory drug is acetaminophen

In an embodiment, the non-steroidal anti-inflammatory drug is naproxen.

In an embodiment, the non-cannabinoid therapeutic compound is the benzodiazepine.

In an embodiment, the benzodiazepine is diazepam.

In an embodiment, the benzodiazepine is alprazolam.

In an embodiment, the benzodiazepine is clonazepam.

In an embodiment, the benzodiazepine is temazepam.

In an embodiment, the cannabinoid compound is encapsulated.

In an embodiment, the non-cannabinoid therapeutic compound is encapsulated.

In an embodiment, the cannabinoid compound and the non-cannabinoid therapeutic compound are encapsulated.

In an embodiment, the cannabinoid compound and the non-cannabinoid therapeutic compound are encapsulated in the same microcapsule of a plurality of microcapsules of the composition.

In an embodiment, the cannabinoid compound and the non-cannabinoid therapeutic compound are encapsulated in different microcapsules of a plurality of microcapsules of the composition.

In an embodiment, the composition is in a solid dosage form.

In an embodiment, the solid dosage form comprises one or more members selected from the group consisting of a pill, tablet, powder, capsule, and granule.

In an embodiment, the cannabinoid compound and the non-cannabinoid therapeutic compound are in the same solid dosage form.

In an embodiment, the cannabinoid compound and the non-cannabinoid therapeutic compound are in different solid dosage forms.

In an embodiment, the composition comprises a first unit dosage form comprising the cannabinoid compound and a second unit dosage form comprising the non-cannabinoid therapeutic compound, wherein the first unit dosage form and the second unit dosage form are for sequential or simultaneous administration to a subject in need thereof.

In an embodiment, the composition is capable of synergistically yielding a greater degree of therapeutic efficacy in a subject in need thereof, as compared to either one of the cannabinoid compound and the non-cannabinoid therapeutic compound alone.

In another aspect, the present disclosure provides a kit comprising the composition of any one of the preceding embodiments, further comprising an instruction for administration of the composition to a subject in need thereof.

In another aspect, the present disclosure provides a method comprising administering the composition of any one of the preceding claims to a subject in need thereof.

In an embodiment, the cannabinoid targets the endocannabinoid system.

In an embodiment, the administration of the composition results in one or more effects from the group consisting of improved therapeutic window, decreased toxicity, decreased side effects, enhanced therapeutic effects, and enhancement of overall homeostasis through improvement in homeostasis in cells, organs, or entire body systems of the subject.

In another aspect, the present disclosure provides a composition comprising a cannabinoid compound and a non-cannabinoid therapeutic compound, wherein the non-cannabinoid therapeutic compound is selected from the group consisting of Enbrel, Neulasta, Prolia, XGEVA, Aranesp, Sensipar/Mimpara, Avsola, Otezla, Parsabiv, AndroGel, Creon, Duopa, Humira, Imbruvica, Niaspan, Orilissa, Skyrizi, Synagis, Trilipix, Advair, Tivicay, Breo/Relvar Ellipta, Ventolin, Lamictal, Avodart, Flovent, Augmentin, Rotarix, Shingrix, Januvia, Janumet, Isentress, Bridion, NuvaRing, Simponi, Zetia/Vytorin, Herceptin, Avastin, Rituxan, Perjeta, Xolair, Actemra, Lucentis, Activase/TNKase, Esbriet, Lantus, Aubagio, Lovenox, Plavix, Myozyme, Toujeo, Dupixent, Fabrazyme, Trulicity, Humalog, Alimta, Cialis, Forteo, Humulin, Taltz, Basaglar, Cymbalta, Xarelto, Eylea, Mirena, Kogenate, Nexavar, Yasmin, Precose, Adalat, Aspirin Cardio, Betaseron, Tagrisso, Symbicort, Brilinta, Farxiga, Nexium, Imfinzi, Pulmicort, Crestor, Lynparza, Faslodex, Eliquis, Opdivo, Sprycel, Orencia SC, Yervoy, Orencia, Revlimid, Baraclude, Empliciti, Nulojix, Stelara, Remicade, Zytiga, Invega Sustenna, Darzalex, Prezista, Imbruvica, Opsumit, Prevnar 13, Lyrica, Ibrance, Lipitor, Xeljanz, Chantix, Sutent, Norvasc, Premarin, Gilenya, Cosentyx, Lucentis, Tasigna, Sandostatin, Gleevec, Afinitor, Galvus, Promacta, Tafinlar, Entyvio, Velcade, Leuprorelin, Azilva, Dexilant, Ninlaro, Genvoya, Truvada, Epclusa, Odefsey, Descovy, Harvoni, Atripla, Biktarvy, Letairis, Ranexa, Copaxone, Bendeka, Methylphenidate Hydrochloride, ProAir HFA, Austedo, QVAR RediHaler, Daptomycin, Metoprolol Succinate, Sildenafil Citrate, Hypochlorous acid, doxorubicin, nano-inducing liposomal doxorubicin, a nano-inducing liposomal taxane, ketamine, a statin, dextromethorphan, a non-steroidal ani-inflammatory drug, and a benzodiazepine.

In an embodiment, at least one of i) the cannabinoid compound and ii) the non-cannabinoid therapeutic compound is encapsulated.

In an embodiment, the non-cannabinoid therapeutic compound is the doxorubicin.

In an embodiment, the non-cannabinoid therapeutic compound is the nano-inducing liposomal doxorubicin.

In an embodiment, the non-cannabinoid therapeutic compound is the nano-inducing liposomal taxane.

In an embodiment, the non-cannabinoid therapeutic compound is the ketamine.

In an embodiment, the non-cannabinoid therapeutic compound is the statin.

In an embodiment, the statin is atorvastatin

In an embodiment, the statin is fluvastatin.

In an embodiment, the statin is lovastatin.

In an embodiment, the non-cannabinoid therapeutic compound is the dextromethorphan.

In an embodiment, the non-cannabinoid therapeutic compound is the non-steroidal ani-inflammatory drug.

In an embodiment, the non-steroidal anti-inflammatory drug is ibuprofen.

In an embodiment, the non-steroidal anti-inflammatory drug is acetaminophen.

In an embodiment, the non-steroidal anti-inflammatory drug is naproxen.

In an embodiment, the non-cannabinoid therapeutic compound is the benzodiazepine.

In an embodiment, the benzodiazepine is diazepam.

In an embodiment, the benzodiazepine is alprazolam.

In an embodiment, the benzodiazepine is clonazepam.

In an embodiment, the benzodiazepine is temazepam.

In an embodiment, the cannabinoid compound is encapsulated.

In an embodiment, the non-cannabinoid therapeutic compound is encapsulated.

In an embodiment, the cannabinoid compound and the non-cannabinoid therapeutic compound are encapsulated.

In an embodiment, the cannabinoid compound and the non-cannabinoid therapeutic compound are encapsulated in the same microcapsule of a plurality of microcapsules of the composition.

In an embodiment, the cannabinoid compound and the non-cannabinoid therapeutic compound are encapsulated in different microcapsules of a plurality of microcapsules of the composition.

In an embodiment, the composition is in a solid dosage form.

In an embodiment, the solid dosage form comprises one or more members selected from the group consisting of a pill, tablet, powder, capsule, and granule.

In an embodiment, the cannabinoid compound and the non-cannabinoid therapeutic compound are in the same solid dosage form.

In an embodiment, the cannabinoid compound and the non-cannabinoid therapeutic compound are in different solid dosage forms.

In an embodiment, the composition comprises a first unit dosage form comprising the cannabinoid compound and a second unit dosage form comprising the non-cannabinoid therapeutic compound, wherein the first unit dosage form and the second unit dosage form are for sequential or simultaneous administration to a subject in need thereof.

In an embodiment, the composition is capable of synergistically yielding a greater degree of therapeutic efficacy in a subject in need thereof, as compared to either one of the cannabinoid compound and the non-cannabinoid therapeutic compound alone.

In another aspect, the present disclosure provides a kit comprising the composition of any one of the preceding embodiments, further comprising an instruction for administration of the composition to a subject in need thereof.

In another aspect, the present disclosure provides a method comprising administering a composition as in any one of the preceding embodiments to a subject in need thereof.

In an embodiment, cannabinoid targets the endocannabinoid system.

In an embodiment, the administration of the composition results in one or more effects from the group consisting of improved therapeutic window, decreased toxicity, decreased side effects, enhanced therapeutic effects, and enhancement of overall homeostasis through improvement in homeostasis in cells, organs, or entire body systems of the subject.

In another aspect, the present disclosure provides a method comprising administering to a subject in need thereof a composition comprising a cannabinoid compound and a non-cannabinoid therapeutic compound, wherein the non-cannabinoid therapeutic compound is selected from the group consisting of Kyprolis, Epogen, Nplate, Vectibix, Blincyto, Corlanor, Evenity, Imlygic, Kanjinti, MVASI, Neupogen, Prolia, Repatha, Depakote, Gengraf, Kaletra, Leuplin, Mavyret, Oriahnn, Rinvoq, Survanta, Synthroid, ultane, venclexta, viekira, zemplar, Triumeq, Keytruda, Gardasil, Pneumovax, Ocrevus, Cyramza, doxorubicin, nano-inducing liposomal doxorubicin, a nano-inducing liposomal taxane, ketamine, a statin, dextromethorphan, a non-steroidal ani-inflammatory drug, a benzodiazepine, and Tables 1-10.

In another aspect, the present disclosure provides a method comprising administering to a subject in need thereof a comprising a cannabinoid compound and a non-cannabinoid therapeutic compound, wherein the non-cannabinoid therapeutic compound is selected from the group consisting of Enbrel, Neulasta, Prolia, XGEVA, Aranesp, Sensipar/Mimpara, Aranesp, Avsola, Otezla, Parsabiv, AndroGel, Creon, Duopa, Humira, Imbruvica, Niaspan, Orilissa, Skyrizi, Synagis, Trilipix, Advair, Tivicay, Breo/Relvar Ellipta, Ventolin, Lamictal, Avodart, Flovent, Augmentin, Rotarix, Shingrix, Januvia, Janumet, Isentress, Bridion, NuvaRing, Simponi, Zetia/Vytorin, Herceptin, Avastin, Rituxan, Perjeta, Xolair, Actemra, Lucentis, Activase/TNKase, Esbriet, Lantus, Aubagio, Lovenox, Plavix, Myozyme, Toujeo, Dupixent, Fabrazyme, Trulicity, Humalog, Alimta, Cialis, Forteo, Humulin, Taltz, Basaglar, Cymbalta, Xarelto, Eylea, Mirena, Kogenate, Nexavar, Yasmin, Precose, Adalat, Asprin Cardio, Betaseron, Tagrisso, Symbicort, Brilinta, Farxiga, Nexium, Imfinzi, Pulmicort, Crestor, Lynparza, Faslodex, Eliquis, Opdivo, Sprycel, Orencia SC, Yervoy, Orencia, Revlimid, Baraclude, Empliciti, Nulojix, Stelara, Remicade, Zytiga, Xarelto, Invega Sustenna, Simponi, Darzalex, Prezista, Imbruvica, Opsumit, Prevnar 13, Lyrica, Ibrance, Enbrel, Lipitor, Xeljanz, Chantix, Sutent, Norvasc, Premarin, Gilenya, Cosentyx, Lucentis, Tasigna, Sandostatin, Gleevec, Afinitor, Galvus, Promacta, Tafinlar, Entyvio, Velcade, Leuprorelin, Azilva, Dexilant, Ninlaro, Genvoya, Truvada, Epclusa, Odefsey, Descovy, Harvoni, Atripla, Biktarvy, Letairis, Ranexa, Copaxone, Bendeka, Methylphenidate Hydrochloride, ProAir HFA, Austedo, Methylphenidate Hydrochloride, QVAR RediHaler, Daptomycin, Metoprolol Succinate, Sildenafil Citrate, Hypochlorous acid, doxorubicin, nano-inducing liposomal doxorubicin, a nano-inducing liposomal taxane, ketamine, a statin, dextromethorphan, a non-steroidal ani-inflammatory drug, and a benzodiazepine.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings or figures (also “FIG.” and “FIGs.” herein), of which.

FIG. 1A shows an example of a droplet generator.

FIG. 1B shows another example of a droplet generator.

FIG. 1C shows an example of a microfluidic structure.

FIG. 2A shows an exemplary microscope image of an unprocessed composition of quillaja extract, hemp oil, and water at 400× magnification;

FIG. 2B shows an exemplary microscope image of an unprocessed composition of quillaja extract, hemp oil, and water at 1000× magnification;

FIG. 3A shows an exemplary microscope image of a microfluidic processed composition of quillaja extract, hemp oil, and water at 400× magnification;

FIG. 3B shows an exemplary microscope image of a microfluidic processed composition of quillaja extract, hemp oil, and water at 400× magnification;

FIG. 3C shows an exemplary microscope image of a microfluidic processed composition of quillaja extract, hemp oil, and water at 1000× magnification;

FIG. 3D shows an exemplary microscope image of a microfluidic processed composition of quillaja extract, hemp oil, and water at 1000× magnification;

FIG. 4A shows an exemplary microscope image of a microfluidic processed composition of quillaja extract, hemp oil, water, and sodium alginate at 1000× magnification;

FIG. 4B shows an exemplary microscope image of a microfluidic processed composition of quillaja extract, hemp oil, water, and sodium alginate at 1000× magnification;

FIG. 4C shows an exemplary microscope image of a microfluidic processed composition of quillaja extract, hemp oil, water, and sodium alginate at 1000× magnification;

FIGS. 5A-5D illustrate example tablets comprising the therapeutic compositions as disclosed herein; and

FIGS. 6A-6D illustrate example pills comprising the therapeutic compositions as disclosed herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about” or “nearly” as used herein generally refers to within +/−10%, 9%, 8%, 7%, 6%, 5%, 4%, 3%, 2%, or 1% of the designated amount.

The term “subject” or “individual,” as used herein, generally refers to a vertebrate, such as a mammal. A subject may be a patient. Mammals can include, but are not limited to, murines, simians, humans, farm animals, sport animals, and pets. Tissues, cells, and their progeny of a biological entity obtained in vivo or cultured in vitro are also encompassed.

The term “treatment” or “treating” generally refers to an approach for obtaining beneficial or desired results including but not limited to a therapeutic benefit. A therapeutic benefit can include a prophylactic benefit. For example, a treatment can comprise administering a composition (e.g., a therapeutic composition) disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “remission of the condition” or “remission of the subject” generally refers to a decrease in the effects or expression of an ailment, disease, condition, or medical problem or condition. Remission of the subject may refer to remission of one or more conditions of the subject. Remission of the condition may refer to mitigation of negative characteristics of the subject or the condition.

The term “effective amount” or “therapeutically effective amount” generally refers to the quantity of a composition as disclosed herein, that is sufficient to result in a desired activity upon administration to a subject in need thereof. In some cases, the term “therapeutically effective” can refer to that quantity of a composition that is sufficient to delay the manifestation, arrest the progression, relieve or alleviate at least one symptom of a disorder treated by the compositions or methods as disclosed herein.

The present disclosure provides therapeutic compositions comprising a plurality of different therapeutic drugs or compounds, and methods for the manufacture, delivery, and use of such compositions. Combination of a plurality of different therapeutic drugs (i.e., co-therapy) can synergistically yield a greater degree of efficacy (e.g., a therapeutic efficacy in a subject in need thereof) as compared to either one of the plurality of different therapeutic drugs alone. Combination of a plurality of different therapeutic drugs (e.g., into a single composition) can promote enhanced medication adherence or compliance by the subject in need thereof as compared to having to take the plurality of different therapeutic drugs separately. In some embodiments, the composition can comprise a first therapeutic compound (e.g., a cannabinoid compound) and a second therapeutic compound (e.g., a non-cannabinoid therapeutic compound) that is different than the first therapeutic compound. The composition can be in the form of a liquid, solid (e.g., pill, tablet, powder, capsule, granule, etc.), semi-solid (e.g., gel), or mixtures thereof.

In an embodiment, the composition can comprise a first composition comprising a first therapeutic compound (e.g., a cannabinoid compound) and second composition comprising a second therapeutic compound (e.g., a non-cannabinoid therapeutic compound). The first composition and the second composition can be in the same unit dose (e.g., the same unit dosage form). Alternatively, the first composition and the second composition can be in different unit doses (e.g., in different unit dosage forms).

In an embodiment, the composition can be a solid composition (e.g., pill, tablet, powder, capsule, granule, etc.). The solid composition can comprise a plurality of first therapeutic compounds (e.g., cannabinoid compounds) and a plurality of second therapeutic compounds (e.g., non-cannabinoid therapeutic compounds).

In some cases, the plurality of first therapeutic compounds and the plurality of second therapeutic compounds can be mixed (e.g., homogenous mixture, non-homogeneous or heterogeneous mixture) within the solid composition.

In some cases, the plurality of first therapeutic compounds and the plurality of second therapeutic compounds may not be mixed. The plurality of first therapeutic compounds and the plurality of second therapeutic compounds may be in different portions of the solid composition. For example, (1) one of the plurality of first therapeutic compounds and the plurality of second therapeutic compounds can be in a core of the solid composition and (2) the other of the plurality of first therapeutic compounds and the plurality of second therapeutic compounds can be in an outer layer (e.g., shell) of the core of the solid composition. In another example, the solid composition can comprise a plurality of layers, and (1) one of the plurality of first therapeutic compounds and the plurality of second therapeutic compounds can be in a first layer of the plurality of layers while (2) the other of the plurality of first therapeutic compounds and the plurality of second therapeutic compounds can be in a second layer of the plurality of layers. The first layer and the second layer can be adjacent to each other. Alternatively, the first layer and the second layer can be separated by at least a third and different layer of the plurality of layers.

Different configurations of the plurality of first therapeutic compounds and the plurality of second therapeutic compounds within the solid composition can provide benefits such as, for example, shelf stability, improved bioavailability, reduced first-pass metabolism, and extended or modified release profiles (e.g., a first release profile of a first drug and a second release profile of a second drug that is different than the first release profile).

In an embodiment, the present disclosure provides for encapsulation of therapeutic compositions as disclosed herein, and methods for the manufacture, delivery, and use of such compositions. At least a portion of the therapeutic compositions can be encapsulated, including in microcapsules. Microencapsulation can provide benefits such as shelf stability, improved bioavailability, reduced first-pass metabolism, and extended or modified release profiles (e.g., a first release profile of a first drug and a second release profile of a second drug that is different than the first release profile). Microencapsulation may involve generating a plurality of droplets in an emulsion. Microencapsulation can increase solubility of the therapeutic compositions in water (e.g., solubility of oil-based therapeutic compositions), such as to ease delivery and/or administration of the therapeutic compositions to a subject. The therapeutic compositions of the present disclosure can comprise cannabinoids, terpenes, essential oils, and other desirable compounds (e.g., non-cannabinoid therapeutic compounds).

In one embodiment, the present disclosure provides a composition comprising a plurality of microcapsules, wherein an individual microcapsule of the plurality comprises one or more therapeutic compositions present in an amount of at least about one microgram.

In some cases, an individual microcapsule of the plurality of microcapsules can comprise a plurality of different therapeutic compounds (e.g., a cannabinoid compound and a non-cannabinoid therapeutic compound). In some cases, a first individual microcapsule of the plurality of microcapsules can comprise a first therapeutic compound (e.g., a cannabinoid compound) and a second individual microcapsule of the plurality of microcapsules can comprise a second therapeutic compound (e.g., a non-cannabinoid therapeutic compound).

In some cases, an individual microcapsule of the plurality of microcapsules of the composition can comprise a cannabinoid compound (i.e., encapsulated cannabinoid compound) and the composition can further comprise a different therapeutic compound (e.g., a non-cannabinoid therapeutic compound) that is not encapsulated. In some cases, an individual microcapsule of the plurality of microcapsules of the composition can comprise a non-cannabinoid therapeutic compound (i.e., encapsulated non-cannabinoid therapeutic compound) and the composition can further comprise a non-encapsulated cannabinoid compound.

In some cases, an individual microcapsule of the plurality of microcapsules can comprise (i) a first therapeutic compound (e.g., a cannabinoid compound) that is encapsulated and (ii) a second therapeutic compound (e.g., a non-cannabinoid therapeutic compound) that is disposed on the surface (e.g., outer surface, inner surface) of the individual microcapsule. In some examples, the individual microcapsule can comprise a plurality of amphiphilic molecules and the second therapeutic compound can be coupled to an amphiphilic molecule of the plurality of amphiphilic molecules (e.g., coupled to a hydrophilic portion of the amphiphilic molecule or a hydrophobic portion of the amphiphilic molecule). In an example, the second therapeutic compound can be coupled to an outer surface of the individual microcapsule.

In some cases, an individual microcapsule of the plurality of microcapsules can comprise a non-cannabinoid therapeutic compound as an emulsifier. The non-cannabinoid therapeutic compound can be an amphiphilic molecule. A plurality of such non-cannabinoid therapeutic compound can be used to form the plurality of microcapsules to encapsulate one or more cannabinoid compounds.

In some embodiments, a non-cannabinoid therapeutic compound as disclosed herein may comprise a non-terpene compound.

Non-limiting examples of a non-cannabinoid therapeutic compound (with indicated therapeutic applications) as disclosed herein can comprise KYPROLIS (or carfilzomib) (e.g., for multiple myeloma), EPOGEN (or epoetin alfa) (e.g., for anemia), Nplate (or Romiplostim) (e.g., for Immune Thromboytopenia), Vectibix (or Panitumumab) (e.g., for Colorectal Cancer), Blincyto (or Blinatumomab) (e.g., for Cancer, such as acute lymphocytic leukemia (ALL)), Corlanor (or Ivabradine) (e.g., heart conditions, such as heart failure), Evenity (or Romosozumab-aqqg) (e.g., for osteoporosis, e.g., in postmenopausal women), Imlygic (or talimogene laherparepvec) (e.g., for melanoma), Kanjinti (or trastuzumab-anns) (e.g., for cancer), MVASI (or bevacizumab) (e.g., for cancer, such as certain colorectal, lung, brain, kidney and cervical cancers), Neupogen (or filgrastim) (e.g., for cancer), Prolia (or Denosumab) (e.g., for osteoporosis), and Repatha (or evolocumab) (e.g., for heart conditions, such as heart attack, stroke, etc.).

Non-limiting examples of a non-cannabinoid therapeutic compound (with indicated therapeutic applications) as disclosed herein can comprise Enbrel (or Etanercept) (e.g., for rheumatoid arthritis), Neulasta (or Filgrastim) (e.g., for chemotherapy), Prolia (or Denosumab) (e.g., for bone cancer, such as osteosarcoma), XGEVA (or Denosumab) (e.g., for bone cancer, such as osteosarcoma), Aranesp (or Darbepoetin alfa) (e.g., for chemotherapy), Sensipar/Mimpara (or Cinacalcet) (e.g., for renal disease), Aranesp (or Darbepoetin alfa) (e.g., for anemia), Avsola (or infliximab-axxq) (e.g., for arthritis), Otezla (or Apremilast) (e.g., for severe plaque psoriasis), and Parsabiv (or etelcalcetide) (e.g., secondary hyperparathyroidism).

Additional non-limiting examples of a non-cannabinoid therapeutic compound (with indicated therapeutic applications) as disclosed herein can comprise AndroGel (or testosterone gel) (e.g., for boosting testosterone), Creon (or pancrelipase) (e.g., for cystic fibrosis), Duopa or (carbidopa/levodopa) (e.g., for Parkinson's disease), Humira (or Adalimumab) (e.g., for Crohn's disease, Plaque Psoriasis, Rheumatoid arthritis), Imbruvica (Ibrutinib) (e.g., for Chronic Lymphocytic Leukemia), Niaspan (or Niacin) (e.g., for Cholesterol Treatment), Orilissa (or elagolix) (e.g., for pain associated with endometriosis), Skyrizi (or risankizumab-rzaa) (e.g., for Plaque Psoriasis), Synagis (or palivizumab) (e.g., for respiratory syncytial virus (RSV)), and Trilipix (or fenofibric acid) (e.g., for High Cholesterol).

Additional non-limiting examples of a non-cannabinoid therapeutic compound (with indicated therapeutic applications) as disclosed herein can comprise Advair (or Fluticasone propionate/Salmeterol) (e.g., for treating asthma and chronic obstructive pulmonary disease (COPD)), Tivicay (or Dolutegravir) (e.g., for antiretroviral threapty, e.g., HIV therapy), Breo/Relvar Ellipta (or fluticasone furoate and vilanterol) (e.g., for treating chronic obstructive pulmonary disease (COPD)), Ventolin (or Albuterol) (e.g., to prevent and/or treat wheezing and shortness of breath caused by breathing problems (e.g., asthma, chronic obstructive pulmonary disease)), Lamictal (or Lamotrigine) (e.g., to prevent and/or control seizures, mood swings of bipolar disorder in adults, etc.), Avodart (or Dutasteride) (e.g., to treat the symptoms of an enlarged prostate (i.e., benign prostatic hyperplasia-BPH)), Flovent (or Fluticasone propionate) (e.g., to treat asthma in patients 4 years and older), Augmentin (or Amoxicillin/Clavulanic acid) (e.g., to treat bacterial infections), Rotarix (or a live attenuated strain of human rotavirus (HRV)) (e.g., for prevention of rotavirus gastroenteritis caused by Gi and non-Gi types), and Shingrix (or recombinant zoster vaccine) (e.g., for Shingles Prophylaxis).

Additional non-limiting examples of a non-cannabinoid therapeutic compound (with indicated therapeutic applications) as disclosed herein can comprise Advair (or Fluticasone propionate/Salmeterol) (e.g., for treating asthma and chronic obstructive pulmonary disease (COPD)), Tivicay (or Dolutegravir) (e.g., for antiretroviral threapty, e.g., HIV therapy), Breo/Relvar Ellipta (or fluticasone furoate and vilanterol) (e.g., for treating chronic obstructive pulmonary disease (COPD)), Ventolin (or Albuterol) (e.g., to prevent and/or treat wheezing and shortness of breath caused by breathing problems (e.g., asthma, chronic obstructive pulmonary disease)), Lamictal (or Lamotrigine) (e.g., to prevent and/or control seizures, mood swings of bipolar disorder in adults, etc.), Avodart (or Dutasteride) (e.g., to treat the symptoms of an enlarged prostate (i.e., benign prostatic hyperplasia-BPH)), Flovent (or Fluticasone propionate) (e.g., to treat asthma in patients 4 years and older), Augmentin (or Amoxicillin/Clavulanic acid) (e.g., to treat bacterial infections), Rotarix (or a live attenuated strain of human rotavirus (HRV)) (e.g., for prevention of rotavirus gastroenteritis caused by Gi and non-Gi types), and Shingrix (or recombinant zoster vaccine) (e.g., for Shingles Prophylaxis).

Additional non-limiting examples of a non-cannabinoid therapeutic compound (with indicated therapeutic applications) as disclosed herein can comprise Depakote (or divalproex sodium) (e.g., for bipolar disorder), Gengraf (or Cyclosporine) (e.g., for preventing or reducing organ rejection in a subject who has received a tissue transplant, such as a liver, kidney, or heart transplant), Kaletra (or lopinavir/ritonavir) (e.g., for HIV), Leuplin (or leuprolide hormone) (e.g., for prostate cancer), Mavyret (or glecaprevir/pibrentasvir) (e.g., for Hepatitis C), Oriahnn (or an estrogen and progestin combination) (e.g., for menstrual bleeding related to uterine fibroids in women before menopause), Rinvoq (or upadacitinib) (e.g., for rheumatoid arthritis), Survanta (or Beractant) (e.g., for Respiratory Distress Syndrome (RDS)), Synthroid (or levothyroxine) (e.g., for Hyperthyroidism), ultane (or sevoflurane) (e.g., for Anesthesia), venclexta (or Venetoclax) (e.g., for chronic lymphocytic leukemia (CLL) or small lymphocytic lymphoma (SLL)), viekira (or ombitasvir, paritaprevir, ritonavir fixed dose combination) (e.g., for hepatitis C virus (HCV)), and zemplar (or paricalcitol) (e.g., for preventing or treating secondary hyperparathyroidism).

Additional non-limiting examples of a non-cannabinoid therapeutic compound (with indicated therapeutic applications) as disclosed herein can comprise Triumeq (or a combination of abacavir, dolutegravir, and lamivudine) (e.g., for HIV).

Additional non-limiting examples of a non-cannabinoid therapeutic compound (with indicated therapeutic applications) as disclosed herein can comprise Januvia (or Sitagliptin) (e.g., for treatment of type 2 diabetes), Janumet (or Sitagliptin/Metformin) (e.g., for Diabetes, Obesity), Isentress (or Raltegravir) (e.g., for HIV/AIDS), Bridion (or Sugammadex) (e.g., for Anaestheis Reversal), active compounds in NuvaRing (or etonogestrel/ethinyl estradiol) (e.g., for Contraception), Simponi (or Golimumab) (e.g., for Rheumatoid arthritis), and Zetia/Vytorin (or Ezetimibe/Simvastatin) (e.g., to treat the symptoms of high cholesterol and to reduce cholesterol).

Additional non-limiting examples of a non-cannabinoid therapeutic compound (with indicated therapeutic applications) as disclosed herein can comprise Keytruda (or Pembrolizumab) (e.g., for non-small-cell lung cancer (NSCLC), melanoma, classical Hodgkin Lymphoma (cHL), urothelial carcinoma, head and neck squamous cell carcinoma (HNSCC), gastric orgastroesophageal junction adenocarcinoma, and microsatellite instability-high (MSI-H) or mismatch repair deficient cancer), Gardasil (or Human Papillomavirus (HPV) Vaccine) (e.g., for HPV), and Pneumovax (or Pneumococcal Vaccination) (e.g., for Pneumococcal disease).

Additional non-limiting examples of a non-cannabinoid therapeutic compound (with indicated therapeutic applications) as disclosed herein can comprise Ocrevus (or ocrelizumab) (e.g., for Relapsing & Remitting MS (PRMS)).

Additional non-limiting examples of a non-cannabinoid therapeutic compound (with indicated therapeutic applications) as disclosed herein can comprise Herceptin (or Trastuzumab) (e.g., for cancer, such as breast cancer), Avastin (or Bevacizumab) (e.g., for advanced colorectal, breast, lung, kidney, cervical and ovarian cancer, relapsed glioblastoma), Rituxan (or Rituximab) (e.g., for blood cancer, rheumatoid arthritis, vasculitis), Perjeta (or Pertuzumab) (e.g., for Breast Cancer Medicine), Xolair (or Omalizumab) (e.g., Asthma), Actemra (or Tocilizumab) (e.g., for Rheumatoid Arthritis), Lucentis (or Ranibizumab) (e.g., Macular Degeneration), Activase/TNKase (or Alteplase/Tenecteplase) (e.g., for Myocardial infarction), and Esbriet (or Pirfenidone) (e.g., Pulmonary fibrosis).

Additional non-limiting examples of a non-cannabinoid therapeutic compound (with indicated therapeutic applications) as disclosed herein can comprise Cyramza (or Ramucirumab) (e.g., for cancer, such as stomach cancer).

Additional non-limiting examples of a non-cannabinoid therapeutic compound (with indicated therapeutic applications) as disclosed herein can comprise Lantus (or Insulin glargine) (e.g., for Diabetes), Aubagio (or teriflunomide) (e.g., for Relapsing Remitting MS (RRMS)), Lovenox (or Enoxaparin sodium) (e.g., for thrombosis), Plavix (or Clopidogrel) (e.g., for Heart Attack, Stroke), Myozyme (or Alglucosidase alfa) (e.g., for Pompe Disease), Toujeo (or Insulin glargine) (e.g., for Diabetes), Dupixent (or Dupilumab) (e.g., Eczema, Dermatitis), and Fabrazyme (or agalsidase beta) (e.g., for Fabry disease).

Additional non-limiting examples of a non-cannabinoid therapeutic compound (with indicated therapeutic applications) as disclosed herein can comprise Trulicity (or Dulaglutide) (e.g., for Diabetes), Humalog (or Insulin lispro) (e.g., for Diabetes), Alimta (or Pemetrexed) (e.g., for Cancer), Cialis (or Tadalafil) (e.g., for Erectile Dysfunction), Forteo (or Teriparatide) (e.g., for Osteoporosis), Humulin (or recombinant human insulin) (e.g., for Diabetes), Taltz (or Ixekizumab) (e.g., for Psoriasis), Basaglar (or Insulin glargine) (e.g., for Diabetes), and Cymbalta (or Duloxetine) (e.g., for Anxiety, Depression).

Additional non-limiting examples of a non-cannabinoid therapeutic compound (with indicated therapeutic applications) as disclosed herein can comprise one or more cancer drugs from Table 1.

TABLE 1

| Types | Therapeutic compounds |
|---|---|
| Chemotherapeutic | Thiotepa, CYTOXAN ® cyclosphosphamide; alkyl sulfonates such as |
| agent | busulfan, improsulfan and piposulfan; aziridines such as benzodopa, |
| | carboquone, meturedopa, and uredopa; ethylenimines and |
| | methylamelamines including altretamine, triethylenemelamine, |
| | trietylenephosphoramide, triethiylenethiophosphoramide and |
| | trimethylolomelamine; acetogenins (especially bullatacin and |
| | bullatacinone); delta-9-tetrahydrocannabinol (dronabinol, |
| | MARINOL ®); beta-lapachone; lapachol; colchicines; betulinic acid; a |
| | camptothecin (including the synthetic analogue topotecan |
| | (HYCAMTIN ®), CPT-11 (irinotecan, CAMPTOSAR ®), |
| | acetylcamptothecin, scopolectin, and 9-aminocamptothecin); |
| | bryostatin; callystatin; CC-1065 (including its adozelesin, carzelesin |
| | and bizelesin synthetic analogues); podophyllotoxin; podophyllinic |
| | acid; teniposide; cryptophycins (particularly cryptophycin 1 and |
| | cryptophycin 8); dolastatin; duocarmycin (including the synthetic |
| | analogues, KW-2189 and CB1-TM1); eleutherobin; pancratistatin; a |
| | sarcodictyin; spongistatin; nitrogen mustards such as chlorambucil, |
| | chlomaphazine, cholophosphamide, estramustine, ifosfamide, |
| | mechlorethamine, mechlorethamine oxide hydrochloride, melphalan, |
| | novembichin, phenesterine, prednimustine, trofosfamide, uracil |
| | mustard; nitrosureas such as carmustine, chlorozotocin, fotemustine, |
| | lomustine, nimustine, and ranimnustine; antibiotics such as the |
| | enediyne antibiotics; dynemicin, including dynemicin A; an |
| | esperamicin; as well as neocarzinostatin chromophore and related |
| | chromoprotein enediyne antiobiotic chromophores), aclacinomysins, |
| | actinomycin, authramycin, azaserine, bleomycins, cactinomycin, |
| | carabicin, carminomycin, carzinophilin, chromomycinis, |
| | dactinomycin, daunorubicin, detorubicin, 6-diazo-5-oxo-L-norleucine, |
| | ADRIAMYCIN ® doxorubicin (including morpholino-doxorubicin, |
| | cyanomorpholino-doxorubicin, 2-pyrrolino-doxorubicin and |
| | deoxydoxorubicin), epirubicin, esorubicin, idarubicin, marcellomycin, |
| | mitomycins such as mitomycin C, mycophenolic acid, nogalamycin, |
| | olivomycins, peplomycin, potfiromycin, puromycin, quelamycin, |
| | rodorubicin, streptonigrin, streptozocin, tubercidin, ubenimex, |
| | zinostatin, zorubicin; anti-metabolites such as methotrexate and 5- |
| | fluorouracil (5-FU); folic acid analogues such as denopterin, |
| | methotrexate, pteropterin, trimetrexate; purine analogs such as |
| | fludarabine, 6-mercaptopurine, thiamiprine, thioguanine; pyrimidine |
| | analogs such as ancitabine, azacitidine, 6-azauridine, carmofur, |
| | cytarabine, dideoxyuridine, doxifluridine, enocitabine, floxuridine; |
| | androgens such as calusterone, dromostanolone propionate, |
| | epitiostanol, mepitiostane, testolactone; anti-adrenals such as |
| | aminoglutethimide, mitotane, trilostane; folic acid replenisher such as |
| | frolinic acid; aceglatone; aidophosphamide glycoside; aminolevulinic |
| | acid; eniluracil; amsacrine; bestrabucil; bisantrene; edatraxate; |
| | defofamine; demecolcine; diaziquone; elfornithine; elliptinium |
| | acetate; an epothilone; etoglucid; gallium nitrate; hydroxyurea; |
| | lentinan; lonidainine; maytansinoids such as maytansine and |
| | ansamitocins; mitoguazone; mitoxantrone; mopidanmol; nitraerine; |
| | pentostatin; phenamet; pirarubicin; losoxantrone; 2-ethylhydrazide; |
| | procarbazine; PSK ® polysaccharide complex (JHS Natural Products, |
| | Eugene, Oreg.); razoxane; rhizoxin; sizofiran; spirogermanium; |
| | tenuazonic acid; triaziquone; 2,2′,2″-trichlorotriethylamine; |
| | trichothecenes (especially T-2 toxin, verracurin A, roridin A and |
| | anguidine); urethan; vindesine (ELDISINE ®, FILDESIN ®); |
| | dacarbazine; mannomustine; mitobronitol; mitolactol; pipobroman; |
| | gacytosine; arabinoside (“Ara-C”); thiotepa; taxoids, for example |
| | taxanes including TAXOL ® paclitaxel (Bristol-Myers Squibb |
| | Oncology, Princeton, N.J.), ABRAXANE ™ Cremophor-free, |
| | albumin-engineered nanoparticle formulation of paclitaxel (American |
| | Pharmaceutical Partners, Schaumberg, Ill.), and TAXOTERE ® |
| | docetaxel (Rhône-Poulenc Rorer, Antony, France); chloranbucil; |
| | gemcitabine (GEMZAR ®); 6-thioguanine; mercaptopurine; |
| | methotrexate; platinum analogs such as cisplatin and carboplatin; |
| | vinblastine (VELBAN ®); platinum; etoposide (VP-16); ifosfamide; |
| | mitoxantrone; vincristine (ONCOVIN ®); oxaliplatin; leucovovin; |
| | vinorelbine (NAVELBINE ®); novantrone; edatrexate; daunomycin; |
| | aminopterin; ibandronate; topoisomerase inhibitor RFS 2000; |
| | difluoromethylornithine (DMFO); retinoids such as retinoic acid; |
| | capecitabine (XELODA ®) |
| Cytotoxic agents | maytansinoids (e.g., DM1); auristatins (e.g., MMAE, MMAF) |
| Anti-estrogens and | tamoxifen (including NOLVADEX ® tamoxifen), EVISTA ® |
| selective estrogen | raloxifene, droloxifene, 4-hydroxytamoxifen, trioxifene, keoxifene, |
| receptor modulators | LY117018, onapristone, and FARESTON ® toremifene; anti- |
| (SERMs) | progesterones; estrogen receptor down-regulators (ERDs); agents that |
| | function to suppress or shut down the ovaries, for example, leutinizing |
| | hormone-releasing hormone (LHRH) agonists such as LUPRON ® |
| | and ELIGARD) leuprolide acetate, goserelin acetate, buserelin acetate |
| | and tripterelin; other anti-androgens such as flutamide, nilutamide and |
| | bicalutamide; and aromatase inhibitors that inhibit the enzyme |
| | aromatase, which regulates estrogen production in the adrenal glands, |
| | such as, for example, 4(5)-imidazoles, aminoglutethimide, |
| | MEGASE ® megestrol acetate, AROMASIN ® exemestane, |
| | formestanie, fadrozole, RIVISOR ® vorozole, FEMARA ® letrozole, |
| | ARIMIDEX ® anastrozole. |
| Bisphosphonates | clodronate (for example, BONEFOS ® or OSTACR), DIDROCAL ® |
| | etidronate, NE-58095, ZOMETA ® zoledronic acid/zoledronate, |
| | FOSAMAX ® alendronate, AREDIA ® pamidronate, SKELID ® |
| | tiludronate, or ACTONEL ® risedronate; as well as troxacitabine (a |
| | 1,3-dioxolane nucleoside cytosine analog); antisense oligonucleotides, |
| | particularly those that inhibit expression of genes in signaling |
| | pathways implicated in abherant cell proliferation, such as, for |
| | example, PKC-alpha, Raf, H-Ras, and epidermal growth factor |
| | receptor (EGFR); vaccines such as THERATOPE ® vaccine and gene |
| | therapy vaccines, for example, ALLOVECTIN ® vaccine, |
| | LEUVECTIN ® vaccine, and VAXID ® vaccine; LURTOTECAN ® |
| | topoisomerase 1 inhibitor; ABARELIX ® rmRH; lapatinib ditosylate |
| | (an ErbB-2 and EGFR dual tyrosine kinase small-molecule inhibitor |
| | also known as GW572016) |
| Antibodies | alemtuzumab (Campath), bevacizumab (AVASTIN ®, Genentech); |
| | cetuximab (ERBITUX ®, Imclone); panitumumab (VECTIBIX ®, |
| | Amgen), rituximab (RITUXAN ®, Genentech/Biogen Idec), |
| | pertuzumab (OMNITARG ®, 2C4, Genentech), trastuzumab |
| | (HERCEPTIN ®, Genentech), tositumomab (Bexxar, Corixia), and the |
| | antibody drug conjugate, gemtuzumab ozogamicin (MYLOTARG ®, |
| | Wyeth). Additional humanized monoclonal antibodies with |
| | therapeutic potential as agents in combination with the compounds of |
| | the invention include: apolizumab, aselizumab, atlizumab, |
| | bapineuzumab, bivatuzumab mertansine, cantuzumab mertansine, |
| | cedelizumab, certolizumab pegol, cidfusituzumab, cidtuzumab, |
| | daclizumab, eculizumab, efalizumab, epratuzumab, erlizumab, |
| | feMzumab, fontolizumab, gemtuzumab ozogamicin, inotuzumab |
| | ozogamicin, ipilimumab, labetuzumab, lintuzumab, matuzumab, |
| | mepolizumab, motavizumab, motovizumab, natalizumab, |
| | nimotuzumab, nolovizumab, numavizumab, ocrelizumab, |
| | omalizumab, palivizumab, pascolizumab, pecfusituzumab, |
| | pectuzumab, pexelizumab, ralivizumab, ranibizumab, reslivizumab, |
| | reslizumab, resyvizumab, rovelizumab, ruplizumab, sibrotuzumab, |
| | siplizumab, sontuzumab, tacatuzumab tetraxetan, tadocizumab, |
| | talizumab, tefibazumab, tocilizumab, toralizumab, tucotuzumab |
| | celmoleukin, tucusituzumab, umavizumab, urtoxazumab, |
| | ustekinumab, visilizumab, anti-interleukin-12 (ABT-874/J695, Wyeth |
| | Research and Abbott Laboratories) |
| Tyrosine kinase | an EGFR-targeting agent (e.g., small molecule, antibody, etc.); small |
| inhibitors | molecule HER2 tyrosine kinase inhibitor such as TAK165 available |
| | from Takeda; CP-724,714, an oral selective inhibitor of the ErbB2 |
| | receptor tyrosine kinase (Pfizer and OSI); dual-HER inhibitors such |
| | as EKB-569 (available from Wyeth) which preferentially binds EGFR |
| | but inhibits both HER2 and EGFR-overexpressing cells; lapatinib |
| | (GSK572016; available from Glaxo-SmithKline), an oral HER2 and |
| | EGFR tyrosine kinase inhibitor; PKI-166 (available from Novartis); |
| | pan-HER inhibitors such as canertinib (CI-1033; Pharmacia); Raf-1 |
| | inhibitors such as antisense agent ISIS-5132 available from ISIS |
| | Pharmaceuticals which inhibit Raf-1 signaling; non-HER targeted TK |
| | inhibitors such as imatinib mesylate (GLEEVEC ®, available from |
| | Glaxo SmithKline); multi-targeted tyrosine kinase inhibitors such as |
| | sunitinib (SUTENT ®, available from Pfizer); VEGF receptor tyrosine |
| | kinase inhibitors such as vatalanib (PTK787/ZK222584, available |
| | from Novartis/Schering AG); MAPK extracellular regulated kinase I |
| | inhibitor CI-1040 (available from Pharmacia); quinazolines, such as |
| | PD 153035, 4-(3-chloroanilino) quinazoline; pyridopyrimidines; |
| | pyrimidopyrimidines; pyrrolopyrimidines, such as CGP 59326, CGP |
| | 60261 and CGP 62706; pyrazolopyrimidines, 4-(phenylamino)-7H- |
| | pyrrolo[2,3-d] pyrimidines; curcumin (diferuloyl methane, 4,5-bis (4- |
| | fluoroanilino)phthalimide); tyrphostines containing nitrothiophene |
| | moieties; PD-0183805 (Warner-Lamber); antisense molecules (e.g., |
| | those that bind to HER-encoding nucleic acid); quinoxalines; |
| | tryphostins; ZD6474 (Astra Zeneca); PTK-787 (Novartis/Schering |
| | AG); pan-HER inhibitors such as CI-1033 (Pfizer); Affinitac (ISIS |
| | 3521; Isis/Lilly); imatinib mesylate (GLEEVEC ®); PKI 166 |
| | (Novartis); GW2016 (Glaxo SmithKline); CI-1033 (Pfizer); EKB-569 |
| | (Wyeth); Semaxinib (Pfizer); ZD6474 (AstraZeneca); PTK-787 |
| | (Novartis/Schering AG); INC-1C11 (Imclone); and rapamycin |
| | (sirolimus, RAPAMUNE ®) |

Additional non-limiting examples of a non-cannabinoid therapeutic compound (with indicated therapeutic applications) as disclosed herein can comprise Xarelto (or Rivaroxaban) (e.g., for Atrial Fibrillation), Eylea (or Aflibercept) (e.g., for Macular Degeneration), active ingredient of Mirena (or levonorgestrel) (e.g., for Contraception), Kogenate (or recombinant antihemophilic factor) (e.g., for Hemophilia), Nexavar (or Sorafenib) (e.g., for cancer), Yasmin (or drospirenone and ethinyl estradiol) (e.g., for contraception), Precose (or Acarbose) (e.g., for Diabetes), Adalat (or Nifedipine) (e.g., for Hypertension), Aspirin Cardio (or aspirin or acetylsalicylic acid) (e.g., for Myocardial infarction prophylaxis), and Betaseron (or Interferon beta-1b) (e.g., multiple sclerosis).

Additional non-limiting examples of a non-cannabinoid therapeutic compound (with indicated therapeutic applications) as disclosed herein can comprise one or more benzodiazepines from Table 2.

TABLE 2

| Type | Therapeutic compounds |
|---|---|
| Benzodi- | alprazolam (Xanax), chlordiazepoxide (Librium), clonazepam |
| azepines | (Klonopin), clorazepate (Tranxene), diazepam (Valium), |
| | estazolam (Prosom), flurazepam (Dalmane), lorazepam |
| | (Ativan), midazolam (Versed), oxazepam (Serax), temazepam |
| | (Restoril), triazolam (Halcion), quazepam (Doral) |

Additional non-limiting examples of a non-cannabinoid therapeutic compound (with indicated therapeutic applications) as disclosed herein can comprise Tagrisso (or Osimertinib) (e.g., for lung cancer), Symbicort (or Budesonide/Formotero) (e.g., for asthma), Brilinta (or Ticagrelor) (e.g., for antiplatelet), Farxiga (or Dapagliflozin) (e.g., for type 2 diabetes), Nexium (or Esomeprazole) (e.g., acid reflux), Imfinzi (or Durvalumab) (e.g., for bladder cancer), Pulmicort (or Budesonide) (e.g., for asthma), Crestor (or Rosuvastatin) (e.g., for cholesterol), Lynparza (or Olaparib) (e.g., for fallopian tube cancer), and Faslodex (or Fulvestrant) (e.g., for breast cancer).

Additional non-limiting examples of a non-cannabinoid therapeutic compound (with indicated therapeutic applications) as disclosed herein can comprise one or more opioid or opioid-mimetic from Table 3.

TABLE 3

| Type | Therapeutic compounds |
|---|---|
| Opioid or | semi-synthetic and synthetic drugs such as hydrocodone, |
| opioid- | oxycodone and fentanyl; antagonist drugs such as naloxone; |
| mimetic | and endogenous peptides such as the endorphins |

Additional non-limiting examples of a non-cannabinoid therapeutic compound (with indicated therapeutic applications) as disclosed herein can comprise Eliquis (or Apixaban) (e.g., for deep vein thrombosis and pulmonary embolism), Opdivo (or Nivolumab) (e.g., for melanoma, lung cancer, renal cancer), Sprycel (or Dasatinib) (e.g., for leukemia), Orencia SC (or abatacept) (e.g., for rheumatoid arthritis), Yervoy (or Ipilimumab) (e.g., for oncology), Orencia (or Abatacept) (e.g., for rheumatoid arthritis), Revlimid (or Lenalidomide) (e.g., for hepatitis B), Baraclude (or Entecavir) (e.g., for HIV/AIDS), Empliciti (or Elotuzumab) (e.g., for multiple myeloma), Nulojix (or Belatacept) (e.g., for kidney transplant).

Additional non-limiting examples of a non-cannabinoid therapeutic compound (with indicated therapeutic applications) as disclosed herein can comprise one or more drugs from Table 4.

TABLE 4

| Type | Therapeutic compounds |
|---|---|
| Drugs | Abiraterone; Abacavir; Abacavir and/or lamivudine; Abiraterone; |
| | Acetazolamide; Acetic acid; Acetylcysteine; Acetylsalicylic acid; |
| | Aciclovir; Activated charcoal; Adalimumab; Afatinib; Albendazole; |
| | Retinoic acid (e.g., trans retinoic acid); Allopurinol; Alteplase; |
| | Amidotrizoate; Amikacin; Amiloride; Amiodarone; Amitriptyline; |
| | Amlodipine; Amodiaquine; Amodiaquine, sulfadoxine, nd/or |
| | pyrimethamine; Amoxicillin; Amoxicillin and/or clavulanic acid; |
| | Amphotericin b; Ampicillin; Anastrozole; Anti-d immunoglobulin; Anti- |
| | rabies immunoglobulin; Anti-tetanus mmunoglobulin; Antivenom |
| | immunoglobulin; Apixaban; Aprepitant; Arsenic trioxide; Artemether; |
| | Artemether and/or lumefantrine; Artesunate; Artesunate and/or |
| | amodiaquine; Artesunate and/or mefloquine; Artesunate and/or |
| | pyronaridine tetraphosphate; Ascorbic acid; Asparaginase; Atazanavir; |
| | Atazanavir and/or ritonavir; Atenolol; Atorvastatin; Atracurium; Atropine; |
| | Azathioprine; Azithromycin; Barium sulfate; Bcg vaccine; Beclometasone; |
| | Bedaquiline; Bendamustine; Benzathine benzylpenicillin; Benznidazole; |
| | Benzoyl peroxide; Benzyl benzoate; Benzylpenicillin; Betamethasone; |
| | Bevacizumab; Bicalutamide; Biperiden; Bisoprolol; Bleomycin; |
| | Bortezomib; Budesonide; Budesonide and/or formoterol; Bupivacaine; |
| | Caffeine citrate; Calamine; Calcium; Calcium folinate; Calcium gluconate; |
| | Capecitabine; Carbamazepine; Carbetocin; Carbimazole; Carboplatin; |
| | Carvedilol; Cefalexin; Cefazolin; Cefixime; Cefotaxime; Ceftazidime; |
| | Ceftazidime and/or avibactam; Ceftriaxone; Cefuroxime; Certolizumab |
| | pegol; Chlorambucil; Chloramphenicol; Chlorhexidine; Chlorine base |
| | compound; Chloroquine; Chloroxylenol; Chlorpromazine; Cholera |
| | vaccine; Ciclosporin; Ciprofloxacin; Cisplatin; Clarithromycin; |
| | Clindamycin; Clofazimine; Clomifene; Clomipramine; Clopidogrel; |
| | Clotrimazole; Cloxacillin; Clozapine; Coagulation factor ix complex; |
| | Coagulation factor viii; Coal tar; Codeine; Colecalciferol; Colistin; |
| | Sodium lactate; Cyclizine; Cyclophosphamide; Cycloserine; Cytarabine; |
| | Dabigatran; Dacarbazine; Daclatasvir; Dactinomycin; Dalteparin; |
| | Dapsone; Darbepoetin alfa; Darunavir; Dasabuvir; Dasatinib; |
| | Daunorubicin; Deferoxamine; Delamanid; Dengue vaccine; Desmopressin; |
| | Dexamethasone; Dextran 70; Diaphragms; Diazepam; Diazoxide; |
| | Diethylcarbamazine; Digoxin; Dihydroartemisinin and/or piperaquine |
| | phosphate; Diloxanide; Dimercaprol; Diphtheria antitoxin; Diphtheria |
| | vaccine; Docetaxel; Docusate sodium; Dolutegravir; Dolutegravir, |
| | lamivudine, and/or tenofovir; Dopamine; Doxorubicin; Doxycycline; |
| | Edoxaban; Efavirenz; Efavirenz, emtricitabine and/or tenofovir; Efavirenz, |
| | lamivudine and/or tenofovir; Eflornithine; Emtricitabine and/or tenofovir; |
| | Enalapril; Enoxaparin, Entecavir; Ephedrine; Epinephrine; Epoetin alfa; |
| | Epoetin beta; Epoetin theta; Ergocalciferol; Ergometrine; Erlotinib; |
| | Erythromycin; Erythropoiesis-stimulating agents; Estradiol cypionate |
| | and/or medroxyprogesterone acetate; Etanercept; Ethambutol; Ethambutol, |
| | isoniazid, pyrazinamide, and/or rifampicin; Ethambutol, isoniazid, and/or |
| | rifampicin; Ethanol; Ethinylestradiol and/or levonorgestrel; |
| | Ethinylestradiol and/or norethisterone; Ethionamide; Ethosuximide; |
| | Etonogestrel; Etoposide; Fentanyl; Ferrous salt; Ferrous salt and/or folic |
| | acid; Fexinidazole; Filgrastim; Fluconazole; Flucytosine; Fludarabine; |
| | Fludrocortisone; Fluorescein; Fluorouracil; Fluoxetine; Fluphenazine; |
| | Fluvastatin; Folic acid; Fomepizole; Fosfomycin; Plasma (e.g., Fresh- |
| | frozen plasma); Furosemide; Gefitinib; Gemcitabine; Gentamicin; |
| | Glecaprevir and/or pibrentasvir; Gliclazide; Glucagon; Glucose; Glucose |
| | and/or sodium hloride; Glutaral; Glyceryl trinitrate; Golimumab; |
| | Griseofulvin; Haemophilus influenzae type b vaccine; Haloperidol; |
| | Halothane; Heparin sodium; Hepatitis a vaccine; Hepatitis b vaccine; HPV |
| | vaccine; Hydralazine; Hydrochlorothiazide; Hydrocortisone; |
| | Hydromorphone; Hydroxocobalamin; Hydroxycarbamide; |
| | Hydroxychloroquine; Hyoscine butylbromide; Hyoscine hydrobromide; |
| | Ibuprofen; Ifosfamide; Imatinib; Indometacin; Infliximab; Influenza |
| | vaccine; Insulin; Intermediate-acting insulin; Intraperitoneal dialysis |
| | solution; Iodine; Iohexol; Ipratropium bromide; Irinotecan; Isoflurane; |
| | Isoniazid; Isoniazid, pyrazinamide, and/or rifampicin; Isoniazid, |
| | pyridoxine, sulfamethoxazole, and/or trimethoprim; Isoniazid and/or |
| | rifampicin; Isosorbide dinitrate; Itraconazole; Ivermectin; Japanese |
| | encephalitis vaccine; Ketamine; Lactulose; Lamivudine; Lamivudine, |
| | nevirapine, and/or zidovudine; Lamivudine and/or zidovudine; |
| | Lamotrigine; Latanoprost; Ledipasvir and/or sofosbuvir; Lenalidomide; |
| | Leuprorelin; Levamisole; Levodopa and/or carbidopa; Levofloxacin; |
| | Levonorgestrel; Levothyroxine; Lidocaine; Lidocaine and/or epinephrine; |
| | Linezolid; Lisinopril and/or amlodipine; Lisinopril and/or |
| | hydrochlorothiazide; Lithium carbonate; Loperamide; Lopinavir and/or |
| | ritonavir; Loratadine; Lorazepam; Losartan; Lovastatin; Lugol's solution; |
| | Magnesium sulfate; Mannitol; Measles vaccine; Mebendazole; |
| | Medroxyprogesterone acetate; Mefloquine; Meglumine antimoniate; |
| | Meglumine iotroxate; Melarsoprol; Melphalan; Meningococcal meningitis |
| | vaccine; Mercaptopurine; Meropenem; Meropenem and/or vaborbactam; |
| | Mesalazine; Mesna; Metformin; Methadone; Methimazole; Methotrexate; |
| | Methoxy polyethylene glycol-epoetin beta; Methyldopa; |
| | Methylprednisolone; Methylthioninium chloride; Metoclopramide; |
| | Metoprolol; Metronidazole; Miconazole; Midazolam; Mifepristone and/or |
| | misoprostol; Miltefosine; Misoprostol; Morphine; Moxifloxacin; |
| | Micronnutrient; Mumps vaccine; Mupirocin; Nadroparin; Naloxone; |
| | Natamycin; Neostigmine; Nevirapine; Niclosamide; Nicotinamide; |
| | Nifedipine; Nifurtimox; Nilotinib; Nitrofurantoin; Nitrous oxide; |
| | Nivolumab; Norethisterone; Normal immunoglobulin; Nystatin; |
| | Ofloxacin; Ombitasvir, paritaprevir, and/or ritonavir; Omeprazole; |
| | Ondansetron; Oseltamivir; Oxaliplatin; Oxamniquine; Oxycodone; |
| | Oxygen; Oxytocin; P-aminosalicylic acid; Paclitaxel; Pancreatic enzymes; |
| | Paracetamol; Paromomycin; Pegaspargase; Pegylated interferon alfa (2a); |
| | Pegylated interferon alfa (2b); Pembrolizumab; Penicillamine; |
| | Pentamidine; Permethrin; Pertussis vaccine; Phenobarbital; |
| | Phenoxymethylpenicillin; Phenytoin; Phytomenadione; Pilocarpine; |
| | Piperacillin and/or tazobactam; Platelets; Plazomicin; Pneumococcal |
| | vaccine; Podophyllotoxin; Podophyllum resin; Poliomyelitis vaccine; |
| | Polygeline; Polymyxin b; Potassium chloride; Potassium ferric |
| | hexacyanoferrate; Potassium iodide; Potassium permanganate; Povidone |
| | iodine; Pravastatin; Praziquantel; Prednisolone; Primaquine; Procaine |
| | benzylpenicillin; Procarbazine; Progesterone vaginal ring; Proguanil; |
| | Propofol; Propranolol; Propylthiouracil; Prostaglandin e1; Prostaglandin |
| | e2; Protamine sulfate; Pyrantel; Pyrazinamide; Pyridostigmine; |
| | Pyridoxine; Pyrimethamine; Quinine; Rabies vaccine; Raltegravir; |
| | Ranitidine; Realgar-indigo naturalis; Red blood cells; Retinol; Ribavirin; |
| | Riboflavin; Rifabutin; Rifampicin; Rifapentine; Risperidone; Ritonavir; |
| | Rituximab; Rivaroxaban; Rotavirus vaccine; Rubella vaccine; Salbutamol; |
| | Salicylic acid; Selenium sulfide; Senna; Silver sulfadiazine; Simvastatin; |
| | Sodium calcium edetate; Sodium chloride; Sodium fluoride; Sodium |
| | hydrogen carbonate; Sodium nitrite; Sodium nitroprusside; Sodium |
| | stibogluconate; Sodium thiosulfate; Sofosbuvir and/or velpatasvir; |
| | Spectinomycin; Spironolactone; Streptokinase; Streptomycin; Succimer; |
| | Sulfadoxine and/or pyrimethamine; Sulfamethoxazole and/or |
| | trimethoprim; Sulfasalazine; Suramin sodium; Surfactant; |
| | Suxamethonium; Tamoxifen; Telmisartan and/or amlodipine; Telmisartan |
| | and/or hydrochlorothiazide; Tenofovir disoproxil fumarate; Terbinafine; |
| | Testosterone; Tetanus vaccine; Tetracaine; Tetracycline; Thalidomide; |
| | Thiamine; Tick-borne encephalitis vaccine; Timolol; Tioguanine; |
| | Tiotropium bromide; Tranexamic acid; Trastuzumab; Triclabendazole; |
| | Tropicamide; Tuberculin; Typhoid vaccine; Ulipristal; Urea; Valaciclovir; |
| | Valganciclovir; Valproic acid; Vancomycin; Varicella vaccine; |
| | Vecuronium; Verapamil; Vinblastine; Vincristine; Vinorelbine; |
| | Voriconazole; Warfarin; Whole blood; Xylometazoline; Yellow fever |
| | vaccine; Zidovudine; Zinc sulfate |

Additional non-limiting examples of a non-cannabinoid therapeutic compound (with indicated therapeutic applications) as disclosed herein can comprise Stelara (or Ustekinumab) (e.g., for psoriasis), Remicade (or Infliximab) (e.g., for rheumatoid arthritis), Zytiga (or Abiraterone acetate) (e.g., for prostate cancer), Xarelto (or Rivaroxaban) (e.g., for deep vein thrombosis, pulmonary embolism), Invega Sustenna (or Paliperidone) (e.g., for schizophrenia), Simponi (or Golimumab) (e.g., for rheumatoid arthritis), Darzalex (or daratumumab) (e.g., for Multiple myeloma), Prezista (or Darunavir) (e.g., for HIV/AIDS), Imbruvica (or Ibrutinib) (e.g., for oncology), and Opsumit (or Macitentan) (e.g., for hypertension).

Additional non-limiting examples of a non-cannabinoid therapeutic compound (with indicated therapeutic applications) as disclosed herein can comprise one or more generic drugs from Table 5.

TABLE 5

| Type | Therapeutic compounds |
|---|---|
| Generic | ACARBOSE; AMLODIPINE AND VALSARTAN; |
| drugs | AMLODIPINE-OLMESARTAN; BUPROPION ER/SR; |
| | CLOPIDOGREL; DULOXETINE; FELODIPINE ER; |
| | FENOFIBRATE; GABAPENTIN; GLYBURIDE/ |
| | METFORMIN; IRBESARTAN; LAMOTRIGINE; |
| | LOSART/HCTZ; METOPROLOL SUC ER; |
| | MIRTAZAPINE; MOEXIPRIL HCL; MONTELUKAST |
| | SOD; PIOGLITAZONE; QUETIAPINE; QUINAPRIL; |
| | RISPERIDONE; ROPINIROLE; ROSUVASTATIN; |
| | TOPIRAMATE; TORSEMIDE; VENLAFAXINE; |
| | VENLAFAXINE ER CP |

Additional non-limiting examples of a non-cannabinoid therapeutic compound (with indicated therapeutic applications) as disclosed herein can comprise Prevnar 13 (or Pneumococcal 13-valent Conjugate Vaccine) (e.g., for pneumococcal vaccine), Lyrica (or Pregabalin) (e.g., for epilepsy, neuropathy), Ibrance (Palbociclib) (e.g., for cancer), Enbrel (or Etanercept) (e.g., for rheumatoid arthritis), Lipitor (or Atorvastatin) (e.g., for cholesterol), Xeljanz (or Tofacitinib) (e.g., for arthritis), Chantix (or Varenicline) (e.g., for smoking cessation), Sutent (or Sunitinib) (e.g., for cancer), Norvasc (or Amlodipine) (e.g., for hypertension), and Premarin (or conjugated estrogens) (e.g., for menopause).

Additional non-limiting examples of a non-cannabinoid therapeutic compound (with indicated therapeutic applications) as disclosed herein can comprise Gilenya (or Fingolimod) (e.g., for autoimmune disease), Cosentyx (or secukinumab) (e.g., for Psoriasis, ankylosing spondylitis and psoriatic arthritis), Lucentis (or Ranibizumab) (e.g., for age-related macular degeneration), Tasigna (or Nilotinib) (e.g., for chronic myeloid leukemia), Sandostatin (or Octreotide) (e.g., for acromegaly), Gleevec (or Imatinib) (e.g., for Leukaemia, chronic myeloid (CML)), Afinitor (or Everolimus) (e.g., for oncology), Galvus (or Vildagliptin) (e.g., for diabetes), Promacta (or Eltrombopag) (e.g., for Thrombocytopaenic purpura or immune thrombocytopenia (ITP)), and Tafinlar (or Dabrafenib) (e.g., for Melanoma).

Additional non-limiting examples of a non-cannabinoid therapeutic compound (with indicated therapeutic applications) as disclosed herein can comprise one or more therapeutic drugs from Table 6.

TABLE 6

| Type | Therapeutic compounds |
|---|---|
| Therapeutic | Atorvastatin; Lisinopril; Metformin Hydrochloride; Amlodipine; |
| drugs | Metoprolol; Albuterol; Omeprazole; Losartan Potassium; Simvastatin; |
| | Gabapentin; Acetaminophen; Hydrocodone Bitartrate; |
| | Hydrochlorothiazide; Sertraline Hydrochloride; Montelukast; Fluticasone; |
| | Amoxicillin; Furosemide; Pantoprazole Sodium; Acetaminophen; |
| | Prednisone; Escitalopram Oxalate; Fluoxetine Hydrochloride; |
| | Dextroamphetamine; Dextroamphetamine Saccharate; Amphetamine; |
| | Amphetamine Aspartate; Tramadol Hydrochloride; Insulin Glargine; |
| | Bupropion; Ibuprofen; Rosuvastatin; Pravastatin Sodium; Trazodone |
| | Hydrochloride; Tamsulosin Hydrochloride; Carvedilol; Meloxicam; |
| | Citalopram; Duloxetine; Alprazolam; Clopidogrel Bisulfate; Aspirin; |
| | Ranitidine; Atenolol; Cyclobenzaprine; Glipizide; Methylphenidate; |
| | Azithromycin; Clonazepam; Oxycodone; Allopurinol; Venlafaxine; |
| | Hydrochlorothiazide; Lisinopril; Warfarin; Propranolol Hydrochloride; |
| | Hydrochlorothiazide; Losartan Potassium; Cetirizine; Estradiol; Ethinyl |
| | Estradiol; Norethindrone; Lorazepam; Quetiapine Fumarate; Zolpidem |
| | Tartrate; Ergocalciferol; Budesonide; Formoterol; Spironolactone; |
| | Ondansetron; Insulin Aspart; Apixaban; Naproxen; Lamotrigine; |
| | Fluticasone Propionate; Salmeterol Xinafoate; Pregabalin; Glimepiride; |
| | Diclofenac; Fenofibrate; Paroxetine; Clonidine; Loratadine; Diltiazem |
| | Hydrochloride; Hydroxyzine; Amitriptyline; Doxycycline; Ethinyl |
| | Estradiol; Norgestimate; Lisdexamfetamine Dimesylate; Sitagliptin |
| | Phosphate; Latanoprost; Cephalexin; Tizanidine; Finasteride; Lovastatin; |
| | Topiramate; Insulin Lispro; Sulfamethoxazole; Trimethoprim; Buspirone |
| | Hydrochloride; Oseltamivir Phosphate; Amoxicillin; Clavulanate |
| | Potassium; Valsartan; Levetiracetam; Hydralazine Hydrochloride; |
| | Mirtazapine; Rivaroxaban; Aripiprazole; Oxybutynin; Esomeprazole; |
| | Alendronate Sodium; Folic Acid; Triamcinolone; Tiotropium; Thyroid; |
| | Ciprofloxacin; Isosorbide Mononitrate; Sumatriptan; Insulin Detemir; |
| | Benzonatate; Famotidine; Diazepam; Ropinirole Hydrochloride; |
| | Hydrochlorothiazide; Triamterene; Benazepril Hydrochloride; |
| | Metronidazole; Methocarbamol; Nifedipine; Baclofen; Methotrexate; |
| | Ezetimibe; Celecoxib; Guanfacine; Donepezil Hydrochloride; |
| | Hydroxychloroquine; Clindamycin; Divalproex Sodium; Morphine; |
| | Ethinyl Estradiol; Levonorgestrel; Prednisolone; Enalapril Maleate; |
| | Pioglitazone; Cyanocobalamin; Norethindrone; Meclizine Hydrochloride; |
| | Valacyclovir; Albuterol Sulfate; Ipratropium Bromide; Docusate; |
| | Liraglutide; Verapamil Hydrochloride; Cefdinir; Nortriptyline |
| | Hydrochloride; Timolol; Dulaglutide; Promethazine Hydrochloride; |
| | Acyclovir; Fluconazole; Methylprednisolone; Metformin Hydrochloride; |
| | Sitagliptin Phosphate; Ramipril; Dexmethylphenidate Hydrochloride; |
| | Brimonidine Tartrate; Oxcarbazepine; Risperidone; Levofloxacin; |
| | Chlorthalidone; Atomoxetine Hydrochloride; Polyethylene Glycol 3350; |
| | Calcium; Cholecalciferol; Mupirocin; Ethinyl Estradiol; Etonogestrel; |
| | Drospirenone; Ethinyl Estradiol; Phentermine; Carbidopa; Levodopa; |
| | Omega-3-acid Ethyl Esters; Desogestrel; Ethinyl Estradiol; Guaifenesin; |
| | Rizatriptan Benzoate; Irbesartan; Doxazosin Mesylate; Linagliptin; |
| | Adalimumab; Nitrofurantoin; Budesonide; Amlodipine Besylate; |
| | Benazepril Hydrochloride; Hydrochlorothiazide; Valsartan; Digoxin; |
| | Acetaminophen; Butalbital; Insulin Degludec; Ketoconazole; Temazepam; |
| | Amiodarone Hydrochloride; Hydrocortisone; Memantine Hydrochloride; |
| | Canagliflozin; Ketorolac Tromethamine; Liothyronine Sodium; Lithium; |
| | Dicyclomine Hydrochloride; Pramipexole Dihydrochloride; Nebivolol |
| | Hydrochloride; Terazosin; Colchicine; Magnesium; Sucralfate; |
| | Medroxyprogesterone Acetate; Glyburide; Carbamazepine; Gemfibrozil; |
| | Nystatin |

Additional non-limiting examples of a non-cannabinoid therapeutic compound (with indicated therapeutic applications) as disclosed herein can comprise one or more cardiac medications from Table 7.

TABLE 7

| Indications | Therapeutic compounds |
|---|---|
| Anticoagulants | Apixaban (Eliquis); Dabigatran (Pradaxa); Edoxaban (Savaysa); |
| | Heparin (various); Rivaroxaban (Xarelto); Warfarin (Coumadin) |
| Antiplatelet Agents and | Aspirin; Clopidogrel (Plavix); Dipyridamole (Persantine); |
| Dual Antiplatelet Therapy | Prasugrel (Effient); Ticagrelor (Brilinta) |
| Angiotensin-Converting | Benazepril (Lotensin); Captopril (Capoten); Enalapril (Vasotec); |
| Enzyme (ACE) Inhibitors | Fosinopril (Monopril); Lisinopril (Prinivil, Zestril); Moexipril |
| | (Univasc); Perindopril (Aceon); Quinapril (Accupril); Ramipril |
| | (Altace); Trandolapril (Mavik) |
| Angiotensin II Receptor | Azilsartan (Edarbi); Candesartan (Atacand); Eprosartan |
| Blockers | (Teveten); Irbesartan (Avapro); Losartan (Cozaar); Olmesartan |
| | (Benicar); Telmisartan (Micardis); Valsartan (Diovan) |
| Angiotensin Receptor- | Sacubitril/valsartan (Entresto) |
| Neprilysin Inhibitors | |
| Beta Blockers | Acebutolol (Sectral); Atenolol (Tenormin); Betaxolol (Kerlone); |
| | Bisoprolol/hydrochlorothiazide (Ziac); Bisoprolol (Zebeta); |
| | Metoprolol (Lopressor, Toprol XL); Nadolol (Corgard); |
| | Propranolol (Inderal); Sotalol (Betapace) |
| Calcium Channel Blockers | Amlodipine (Norvasc); Diltiazem (Cardizem, Tiazac); |
| | Felodipine (Plendil); Nifedipine (Adalat, Procardia); |
| | Nimodipine (Nimotop); Nisoldipine (Sular); Verapamil (Calan, |
| | Verelan) |
| Cholesterol-lowering | Atorvastatin (Lipitor); Fluvastatin (Lescol); Lovastatin |
| medications | (Mevacor); Pitavastatin (Livalo); Pravastatin (Pravachol); |
| | Rosuvastatin (Crestor); Simvastatin (Zocor); Nicotinic acids; |
| | Ezetimibe (Zetia); Ezetimibe/Simvastatin (Vytorin) |
| Digitalis Preparations | Digoxin (Lanoxin) |
| Diuretics | Acetazolamide (Diamox); Amiloride (Midamor); Bumetanide |
| | (Bumex); Chlorothiazide (Diuril); Chlorthalidone (Hygroton); |
| | Furosemide (Lasix); Hydro-chlorothiazide (Esidrix, |
| | Hydrodiuril); Indapamide (Lozol); Metalozone (Zaroxolyn); |
| | Spironolactone (Aldactone); Torsemide (Demadex) |
| Vasodilators | Isosorbide dinitrate (Isordil); Isosorbide mononitrate (Imdur); |
| | Hydralazine (Apresoline); Nitroglycerin (Nitro Bid, Nitro Stat); |
| | Minoxidil |

Additional non-limiting examples of a non-cannabinoid therapeutic compound (with indicated therapeutic applications) as disclosed herein can comprise Entyvio (or Vedolizumab) (e.g., for ulcerative colitis, Crohn's disease), Velcade (Bortezomib) (e.g., for multiple myeloma), Leuprorelin (or leuprolide) (e.g., for prostate cancer, breast cancer, endometriosis), Azilva (or azilsartan medoxomil) (e.g., for hypertension), Dexilant (or Dexlansoprazole) (e.g., for acid reflux), and Ninlaro (or Ixazomib) (e.g., for multiple myeloma).

Additional non-limiting examples of a non-cannabinoid therapeutic compound (with indicated therapeutic applications) as disclosed herein can comprise one or more gastrointestinal agents from Table 8.

TABLE 8

| Indications | Therapeutic compounds |
|---|---|
| Gastrointestinal | 5-aminosalicylates; antacids; antidiarrheals; digestive |
| agents | enzymes; functional bowel disorder agents (e.g., |
| | anticholinergics/antispasmodics; chloride channel |
| | activators; guanylate cyclase-C agonists; NHE3 |
| | inhibitors; peripheral opioid receptor antagonists; |
| | peripheral opioid receptor mixed agonists/antagonists; |
| | serotoninergic neuroenteric modulators); gallstone |
| | solubilizing agents; GI stimulants; H. pylori eradication |
| | agents; H2 antagonists; Laxatives; miscellaneous GI |
| | agents; proton pump inhibitors |

Additional non-limiting examples of a non-cannabinoid therapeutic compound (with indicated therapeutic applications) as disclosed herein can comprise Genvoya (or a combination of elvitegravir, cobicistat, emtricitabine, and tenofovir alafenamide) (e.g., for HIV treatment), Truvada (or Emtricitabine/Tenofovir) (e.g., for HIV treatment), Epclusa (or Sofosbuvir/Velpatasvir) (e.g., for Pre-exposure prophylaxis (PrEP)), Odefsey (or a combination of emtricitabine, rilpivirine, and tenofovir alafenamide) (e.g., for Hepatitis C treatment), Descovy (or Emtricitabine/Tenofovir) (e.g., for Liver cirrhosis), Harvoni (or ledipasvir/sofosbuvir) (e.g., for HIV treatment), Atripla (or Efavirenz/Emtricitabine/Tenofovir) (e.g., for HIV treatment), Biktarvy (or bictegravir, emtricitabine, and tenofovir alafenamide) (e.g., for Hepatitis C treatment), Letairis (or Ambrisentan) (e.g., for HIV treatment), and Ranexa (or Ranolazine) (e.g., for HIV treatment).

Additional non-limiting examples of a non-cannabinoid therapeutic compound (with indicated therapeutic applications) as disclosed herein can comprise one or more steroids from Table 9.

TABLE 9

| Indications | Therapeutic compounds |
|---|---|
| Corticosteroid | Hydrocortisone, Hydrocortisone acetate, Cortisone acetate, |
| | tixocortol pivalate, prednisolone, methylprednisolone, |
| | prednisone, Amcinonide, budesonide, desonide, fluocinolone |
| | acetonide, fluocinonide, halcinonide, and triamcinolone |
| | acetonide, Beclometasone, betamethasone, dexamethasone, |
| | fluocortolone, halometasone, and mometasone, Alclometasone |
| | dipropionate, betamethasone dipropionate, betamethasone |
| | valerate, clobetasol propionate, clobetasone butyrate, |
| | fluprednidene acetate, and mometasone furoate, Ciclesonide, |
| | cortisone acetate, hydrocortisone aceponate, hydrocortisone |
| | acetate, hydrocortisone buteprate, hydrocortisone butyrate, |
| | hydrocortisone valerate, prednicarbate, and tixocortol pivalate |
| Sex steroid | Progestogens (e.g., progesterone), Androgens (e.g., testosterone), |
| | Estrogens (e.g., estradiol) |
| Neurosteroid | tetrahydrodeoxycorticosterone (THDOC), androstane 3α- |
| | androstanediol, cholestane cholesterol, pregnanes pregnanolone |
| | (eltanolone), allopregnanolone (3α,5α-THP), pregnenolone |
| | sulfate (PS), epipregnanolone, isopregnanolone (sepranolone), |
| | androstanes dehydroepiandrosterone (DHEA; prasterone), |
| | dehydroepiandrosterone sulfate (DHEA-S; prasterone sulfate), |
| | cholestane 24(S)-hydroxycholesterol (NMDA receptor- |
| | selective), androstadienol, androstadienone, androstenol, and |
| | androstenone, estrane estratetraenol, pregnenolone, progesterone, |
| | estradiol, corticosterone |
| Aminosteroid | candocuronium iodide (chandonium iodide), dacuronium |
| | bromide, dihydrochandonium, dipyrandium, malouetine, |
| | pancuronium bromide, pipecuronium bromide, rapacuronium |
| | bromide, rocuronium bromide, stercuronium iodide, and |
| | vecuronium bromide |
| Secosteroid | Cholecalciferol |

Additional non-limiting examples of a non-cannabinoid therapeutic compound (with indicated therapeutic applications) as disclosed herein can comprise Copaxone (or Glatiramer acetate) (e.g., for Relapsing-Remitting MS), Bendeka (or Bendamustine) (e.g., for Leukaemia, chronic lymphocytic Non-Hodgkin lymphoma), Methylphenidate Hydrochloride (e.g., for Attention deficit disorder/hyperactivity (ADD/ADHD)), ProAir HFA (or Albuterol) (e.g., for Asthma), Austedo (or deutetrabenazine) (e.g., for Huntington's disease, Tardive dyskinesia), Methylphenidate Hydrochloride (e.g., for Attention deficit disorder/hyperactivity (ADD/ADHD)), QVAR RediHaler (or beclomethasone-dipropionate HFA) (e.g., for asthma), Cubicin (or Daptomycin) (e.g., for Skin infections, Endocarditis), Metoprolol Succinate (e.g., for Hypertension), and Sildenafil Citrate (e.g., for Erectile dysfunction).

Additional non-limiting examples of a non-cannabinoid therapeutic compound (with indicated therapeutic applications) as disclosed herein can comprise one or more over the counter (OTC) health products from Table 10.

TABLE 10

| Indications | Therapeutic compounds |
|---|---|
| Acid reducers | Prilosec (or Omeprazole) |
| Acne products | Differin Gel (or Adapalene) |
| Antibacterial soaps | Hibiclens (or Chlorhexidine) |
| Antidiarrheals | Imodium (or Loperamide) |
| Antihistamines for allergies | Zyrtec (or Cetirizine) |
| Anti-inflammatory products | Advil (or Ibuprofen) |
| Arthritis and joint pain topical | Biofreeze (or menthol) |
| Aspirin for heart health | Bayer (or Aspirin) |
| Children's allergy medicine | Children's Claritin (or Loratadine) |
| Children's cough medicine | Children's Delsym (or Dextromethorphan) |
| Children's multivitamins | Flintstones Complete Children's Multivitamin |
| Children's pain relief | Children's Tylenol (or Acetaminophen) |
| Cholesterol management | Nature Made Fish Oil |
| Cold remedies | Cepacol (or one or more of cetylpyridinium chloride, |
| | benzocaine, and menthol) |
| Cold sore treatments | Abreva (or Docosanol) |
| Cough suppressants | Delsym (or Dextromethorphan) |
| Diabetic foot cream | Eucerin (or one or more of Olamine, Caprylyl Glycol, |
| | Ethylhexylglycerin, Benzyl Alcohol, and Citric Acid) |
| Eye drops for allergy relief | Zaditor (or Ketotifen) |
| Fiber Laxatives | Metamucil (or psyllium husk) |
| Flu treatment | DayQuil Cold & Flu (or one or more of |
| | Acetaminophen, Dextromethorphan HBr, and |
| | Phenylephrine HCl) |
| Foot care products | Gold Bond (or Menthol) |
| Headache relief | Advil (or Ibuprofen) |
| Insect bite and sting management | After Bite (or Sodium Bicarbonate) |
| Lactose intolerance products | Lactaid (or Lactase Enzyme) |
| Leg cramp relief | Hyland's Leg Cramps (or one or more of Aconitum |
| | Nap., Cinchona, Gnaphalium Poly., Ledum, Mag. |
| | Phos., Rhus Tox., and Viscum) |
| Lice treatments | Nix (or Viscum album) |
| Lip balm | Carmex (or benzocaine) |
| Nausea remedies | Emetrol (or one or more of dextrose, levulose, and |
| | phosphoric acid) |
| Menstrual pain relief | Midol Complete (or one or more of acetaminophen, |
| | caffeine, and pyrilamine maleate) |
| Migraine relief | Excedrin Migraine (or one or more of acetaminophen, |
| | aspirin and caffeine) |
| Motion sickness remedies | Dramamine (or Dimenhydrinate) |
| Oral decongestants | Sudafed (or Pseudoephedrine) |
| Scar treatments | Medermas (or Allium cepa) |
| Sleep aid | Tylenol PM (or a combination of Acetaminophen and |
| | Diphenhydramine HCl) |
| Smoking cessation aids | NicoDerm CQ Patch (or nicotine with ethylene vinyl |
| | acetate copolymer and/or polyisobutylene) |
| Sore throat products | Chloraseptic (or phenol) |
| Sunburn relief | Solarcaine (or one or both of Lidocaine hydrochloride |
| | and Aloe Barbadensis Leaf Juice) |
| Toothache products | Orajel (or Benzocaine) |
| Toothpaste for general use | Crest (or Cetylpyridinium chloride) |
| Toothpaste for sensitive gums and | Sensodyne (or stannous fluoride or potassium nitrate) |
| teeth | |
| Topical antibiotics and antiseptics | Neosporin (or one or more of neomycin, polysporin, |
| | and bacitracin) |
| Upset stomach remedies | Pepto-Bismol (or Bismuth subsalicylate) |
| Urinary tract pain relief | AZO Urinary Pain Relief (or Phenazopyridine |
| | Hydrochloride) |
| Wart removers | Compound W (or Salicylic acid) |
| Yeast infection prevention and | Monistat (or Miconazole) |
| relief | |

Additional non-limiting examples of a non-cannabinoid therapeutic compound (with indicated therapeutic applications) as disclosed herein can comprise Hypochlorous acid (HOCL) (e.g., for blepharitis).

Non-limiting examples of conditions of diseases that can be ameliorated or treated with the non-cannabinoid therapeutic compound as disclosed herein can include rheumatoid arthritis, various cancers or tumors (e.g., breast cancer, bone cancer (such as osteosarcoma), colorectal cancer, lung cancer, kidney cancer, cervical cancer, ovarian cancer, relapsed glioblastoma, blood cancer, fallopian tube cancer, renal cancer, prostate cancer), renal disease, anemia, arthritis, severe plaque psoriasis, secondary hyperparathyroidism, testosterone deficiency, cystic fibrosis, Parkinson's disease, Crohn's disease, Plaque Psoriasis, Chronic Lymphocytic Leukemia, Cholesterol conditions (e.g., high cholesterol), pain associated with endometriosis, Plaque Psoriasis, viral infections (e.g., respiratory syncytial virus (RSV), DIV/AIDS), asthma, chronic obstructive pulmonary disease (COPD), wheezing, shortness of breath, chronic obstructive pulmonary disease, seizures, mood swings, bipolar disorder, benign prostatic hyperplasia-BPH), bacterial infections, rotavirus gastroenteritis, Shingles Prophylaxis, diabetes (e.g., type 1 or type 2 diabetes), Obesity, vasculitis, Macular Degeneration, Myocardial infarction, Pulmonary fibrosis, Relapsing Remitting MS (RRMS), thrombosis, Heart Attack, Stroke, Pompe Disease, Eczema, Dermatitis, Fabry disease, Erectile Dysfunction, Osteoporosis, Psoriasis, Anxiety, Depression, Atrial Fibrillation, Hemophilia, Hypertension, Myocardial infarction prophylaxis, multiple sclerosis, acid reflux, deep vein thrombosis, pulmonary embolism, melanoma, leukemia, hepatitis B, multiple myeloma, psoriasis, deep vein thrombosis, pulmonary embolism, schizophrenia, pneumococcal vaccine, epilepsy, neuropathy, addiction (e.g., drug, alcohol, or smoking addiction), menopause, autoimmune disease, Psoriasis, ankylosing spondylitis, psoriatic arthritis, macular degeneration, chronic myeloid leukemia, acromegaly, chronic myeloid (CML), Thrombocytopenic purpura or immune thrombocytopenia (ITP), ulcerative colitis, Crohn's disease, endometriosis, acid reflux, Pre-exposure prophylaxis (PrEP), Hepatitis C treatment, Liver cirrhosis, Non-Hodgkin lymphoma, Attention deficit disorder/hyperactivity (ADD/ADHD), Tardive dyskinesia, Skin infections, and Endocarditis.

Non-limiting examples of conditions of diseases that can be ameliorated or treated with the non-cannabinoid therapeutic compound as disclosed herein can include central nervous system (CNS) disease, such as addiction, arachnoid cyst, attention deficit/hyperactivity disorder (ADHS), autism, bipolar disorder, catalepsy, depression, encephalitis, epilepsy/seizures, infection, Locked-in syndrome, meningitis, migraine, multiple sclerosis, myelopathy, neurodegenerative disorders such as Alzheimer's, Huntington's, or Parkinson's, etc. In some embodiments, the non-cannabinoid therapeutic compound as disclosed herein can be used as chemotherapeutic agent, anesthesia reversal agent, contraception agent, antiplatelet agent, post-tissue transplant medication (e.g., kidney transplant), or vaccine.

In some embodiments, a composition of the present disclosure comprising a cannabinoid and a non-cannabinoid therapeutic compound may be administered to a subject. The subject may have or be suspected of having a condition (e.g., cancer). The condition of the subject may be cancer of the breast, lung, stomach, ovary, or other organ or tissue. In some embodiments, the non-cannabinoid therapeutic compound may comprise or be doxorubicin. Subsequent to the administration, the subject may be characterized by at least a partial remission of the condition. For example, subsequent to administration of a composition of the present disclosure comprising a cannabinoid and a non-cannabinoid therapeutic compound comprising doxorubicin, the subject may be characterized by at least partial mitigation or remission of the cancer. The composition comprising a cannabinoid and doxorubicin (a non-cannabinoid therapeutic) may mitigate the negative side effects of the doxorubicin in the subject. The co-therapeutic composition comprising a cannabinoid and doxorubicin may mitigate cardiomyopathy during treatment of cancer by activating, binding, or otherwise interacting with endocannabinoid receptors in the heart. Alternatively, or in addition, the co-therapeutic composition may mitigate cardiomyopathy during treatment of cancer via mitochondrial pathways. Administration of the composition comprising a cannabinoid in addition to a non-cannabinoid therapeutic compounds, such as doxorubicin, may result in an improved patient outcome compared to administration either the cannabinoid or non-cannabinoid therapeutic compound individually.

The structure of doxorubicin may be

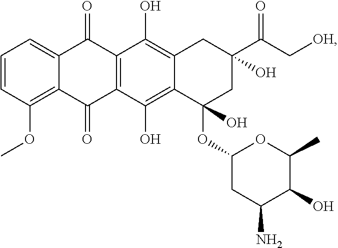 [SMILES: COc1cccc2c1C(=O)c1c(O)c3c(c(O)c1C2=O)C[C@@](O)(C(=O)CO)C[C@]3(O)O[C@H]1C[C@H](N)[C@H](O)[C@H](C)O1]

or a salt, stereoisomer, or prodrug thereof.

In some embodiments, a co-therapeutic composition of the present disclosure comprising a cannabinoid and a non-cannabinoid therapeutic compound may be administered to a subject. The subject may have or be suspected of having a condition, which treatment of the condition may comprise administration of the non-cannabinoid therapeutic compound (e.g., ketamine). In some cases, treatment of the condition by administering the co-therapeutic composition of the present disclosure may improve the treatment of the condition. In some embodiments, the non-cannabinoid therapeutic compound may comprise or be ketamine. Subsequent to the administration, the subject may be characterized by at least a partial remission of the condition. Alternatively, or in addition, subsequent to the administration of the composition comprising the cannabinoid and the non-cannabinoid therapeutic, the subject may be characterized by a least a partial reduction of negative side-effects as compared to administration of the non-cannabinoid therapeutic compound individually. For example, subsequent to administration of a composition comprising a cannabinoid and ketamine, the subject may be characterized by one or more of diminished motor disturbance, antidepressant effects, and support of fear extinction in neural networks.

The structure of ketamine may be

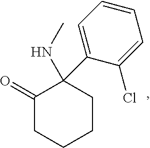 [SMILES: CNC1(c2ccccc2Cl)CCCCC1=O]

or a salt, stereoisomer, or prodrug thereof.

In some embodiments, a co-therapeutic composition of the present disclosure comprising a cannabinoid and a non-cannabinoid therapeutic compound may be administered to a subject. The subject may have or be suspected of having a condition (e.g. high cholesterol). In some cases, the condition may be a condition that is treated through administration of a statin. In some embodiments, the non-cannabinoid therapeutic compound may comprise or be a statin (e.g., atorvastatin, Fluvastatin, pravastatin, rosuvastatin, simvastatin). Subsequent to the administration of the composition comprising a cannabinoid and a non-cannabinoid therapeutic compound (e.g., a statin), the subject may be characterized by at least a partial remission of the condition. Subsequent to administration, the subject may be characterized by improved (e.g., decreased) cholesterol levels and/or neuroprotective effects from the administration of the co-therapeutic composition.

The structure of atorvastatin may be

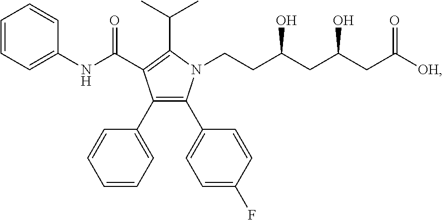 [SMILES: CC(C)c1c(C(=O)Nc2ccccc2)c(-c2ccccc2)c(-c2ccc(F)cc2)n1CC[C@@H](O)C[C@@H](O)CC(=O)O]

or a salt, stereoisomer, or prodrug thereof.

The structure of fluvastatin may be

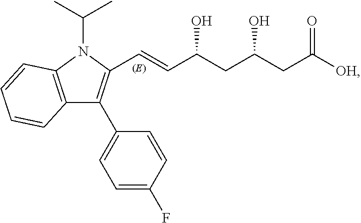 [SMILES: CC(C)n1c(/C=C/[C@H](O)C[C@H](O)CC(=O)O)c(-c2ccc(F)cc2)c2ccccc21]

or a salt, stereoisomer, or prodrug thereof.

The structure of lovastatin may be

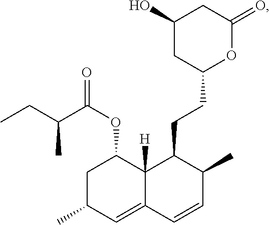 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

or a salt, stereoisomer, or prodrug thereof.

In some embodiments, a co-therapeutic composition of the present disclosure comprising a cannabinoid and a non-cannabinoid therapeutic compound may be administered to a subject. The subject may have or be suspected of having a condition, which treatment of the condition may comprise administration of the non-cannabinoid therapeutic compound. In some cases, the treatment of the condition may be improved by administration of the co-therapeutic compound comprising a cannabinoid and the non-cannabinoid therapeutic. In some embodiments, the non-cannabinoid therapeutic compound may comprise or be dextromethorphan. Subsequent to the administration, the subject may be characterized by at least a partial remission of the condition. As an example, a subject may have a condition that may be mitigated through administering dextromethorphan (e.g., a cough). Subsequent to administration of the co-therapeutic composition comprising a cannabinoid and dextromethorphan, the subject may be characterized by decreased inflammation and enhanced liver protection. Administering the co-therapeutic composition comprising a cannabinoid and dextromethorphan may improve the treatment or reduce one or more negative effects of the non-cannabinoid therapeutic. In some cases, administration of the co-therapeutic composition may improve the treatment of the cough of the subject by decreasing inflammation. In some cases, administration of the co-therapeutic composition comprising a cannabinoid and dextromethorphan provides enhanced liver protection compared to administration of dextromethorphan individually.

The structure of dextromethorphan may be

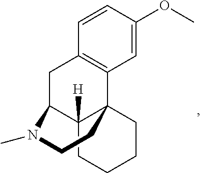

or a salt, stereoisomer, or prodrug thereof.

In some embodiments, a co-therapeutic composition of the present disclosure comprising a cannabinoid and a non-cannabinoid therapeutic compound may be administered to a subject. The subject may have or be suspected of having a condition, which treatment of the condition may comprise administration of the non-cannabinoid therapeutic compound. In some cases, the treatment of the condition may be improved by administration of the co-therapeutic compound comprising a cannabinoid and the non-cannabinoid therapeutic. In some embodiments, the non-cannabinoid therapeutic compound may comprise or be an NSAID (e.g., ibuprofen, acetaminophen, or naproxen). Subsequent to the administration, the subject may be characterized by at least a partial remission of the condition. As an example, a subject may have a condition (e.g. pain, inflammation, fever, headache, or arthritis) that may be mitigated through administering an NSAID. Subsequent to administration of the co-therapeutic composition comprising a cannabinoid and an NSAID, the subject may be characterized by decreased inflammation and enhanced liver protection. Administering the co-therapeutic composition comprising a cannabinoid and an NSAID may improve the treatment or reduce one or more negative effects of the non-cannabinoid therapeutic. In some cases, administration of the co-therapeutic composition may improve the treatment of the condition by decreasing inflammation. In some cases, administration of the co-therapeutic composition comprising a cannabinoid and an NSAID provides enhanced liver protection compared to administration of an NSAID individually.

The structure of ibuprofen may be

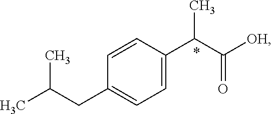

or a salt, stereoisomer, or prodrug thereof.

The structure of acetaminophen may be

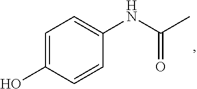

or a salt, isomer, or prodrug thereof.

The structure of naproxen may be

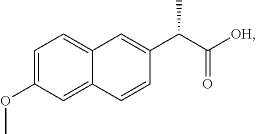 [SMILES: COc1ccc2cc([C@H](C)C(=O)O)ccc2c1]

or a salt, stereoisomer, or prodrug thereof.

In some embodiments, a co-therapeutic composition of the present disclosure comprising a cannabinoid and a non-cannabinoid therapeutic compound may be administered to a subject. The subject may have or be suspected of having a condition, which treatment of the condition may comprise administration of the non-cannabinoid therapeutic compound. In some embodiments, the non-cannabinoid therapeutic compound may comprise or be a benzodiazepine (e.g., alprazolam, estazolam, flunitrazepam, clonazepam, lormetazepam, midazolam, lorazepam, nitrazepam, and temazepam, chlordiazepoxide, clorazepate, diazepam, lorazepam, halazepam, oxazepam, prazepam, or quazepam). Subsequent to the administration, the subject may be characterized by at least a partial remission of the condition. As an example, a subject may have a condition (e.g., anxiety, panic attacks, depression, insomnia, seizures, or naseua) that may be mitigated through administration of a benzodiazepine. Subsequent to administration of the co-therapeutic composition comprising a cannabinoid and a benzodiazepine, the subject may be characterized by one or more of improved anxiolytic effects, lower addiction potential, and protection of the subject's liver and brain as compared to administration of the benzodiazepine individually.

The structure of diazepam may be

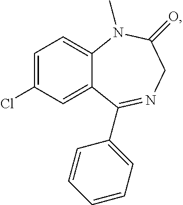 [SMILES: CN1C(=O)CN=C(c2ccccc2)c2cc(Cl)ccc21]

or a salt, isomer, or prodrug thereof.

The structure of alprazolam may be

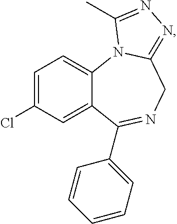 [SMILES: Cc1nnc2n1-c1ccc(Cl)cc1C(c1ccccc1)=NC2]

or a salt, isomer, or prodrug thereof.

The structure of clonazepam may be

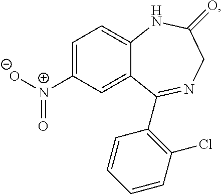 [SMILES: O=C1CN=C(c2ccccc2Cl)c2cc([N+](=O)[O-])ccc2N1]

or a salt, isomer, or prodrug thereof.

The structure of temazepam may be

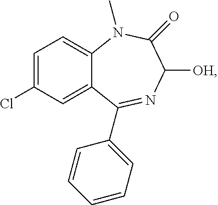 [SMILES: CN1C(=O)C(O)N=C(c2ccccc2)c2cc(Cl)ccc21]

or a salt, isomer, or prodrug thereof.

Without wishing to be bound by theory, the co-therapy of (i) one or more cannabinoid compounds and (ii) one or more non-cannabinoid therapeutic compounds, as disclosed herein, can promote or cause a unique therapeutic effect (e.g., a synergistic effect, or a different effect not elicited by either (i) or (ii) alone in a subject). For example, the co-therapy can enhance efficacy (e.g., therapeutic efficacy) of (i) and/or (ii) by at least about 5%, 10%, 20%, 30%, 40%, 50%, 60%, 70%, 80%, 90%, 100%, 150%, 200%, or more, as compared to treatment with either (i) or (ii) alone. In another example, the co-therapy can elicit a therapeutic pathway (e.g., a cellular signaling pathway) that is not activated by treatment with either (i) or (ii) alone.

Without wishing to be bound by theory, the co-therapy of (i) one or more cannabinoid compounds and (ii) one or more non-cannabinoid therapeutic compounds, as disclosed herein, can enhance efficacy (i.e., enhance overall outcome) of either (i) or (ii) alone in a subject.

Without wishing to be bound by theory, the co-therapy of (i) one or more cannabinoid compounds and (ii) one or more non-cannabinoid therapeutic compounds, as disclosed herein, can enhance therapeutic window (e.g., a range of drug dosages which can treat disease effectively without having toxic effects) of either alone or both of (i) or (ii) in a subject. For example, the co-therapy can increase the therapeutic window of the one or more non-cannabinoid therapeutic compounds in the subject by at least about 5%, 10%, 20%, 30%, 40%, 50%, 60%, 70%, 80%, 90%, 100%, 150%, 200%, or more, as compared to treatment with the one or more non-cannabinoid therapeutic compounds in absence of the one or more cannabinoid compounds.

Without wishing to be bound by theory, the co-therapy of (i) one or more cannabinoid compounds and (ii) one or more non-cannabinoid therapeutic compounds, as disclosed herein, can delay, reduce, or prevent side effects (e.g., off-target effects, toxicity, addiction, etc.) of either (i) or (ii) alone in a subject. For example, the co-therapy can reduce side effects of the one or more non-cannabinoid therapeutic compounds in the subject by at least about 5%, 10%, 20%, 30%, 40%, 50%, 60%, 70%, 80%, 90%, or 95%, or more, as compared to treatment with the one or more non-cannabinoid therapeutic compounds in absence of the one or more cannabinoid compounds.

In another embodiment, the present disclosure provides a composition comprising a plurality of microcapsules, wherein an individual microcapsule of the plurality comprises one or more therapeutic compositions, and wherein the microcapsules are not liposomes or micelles.

In another embodiment, the present disclosure provides a composition comprising a plurality of microcapsules, wherein an individual microcapsule of the plurality of microcapsules comprises one or more therapeutic compositions, and wherein the composition has a shelf half-life of at least 30 days. In other embodiments, the present disclosure provides food products that are rich in therapeutic compositions.

### Microfluidic Encapsulation and Delivery

The compositions of the present disclosure can comprise microcapsules. Microcapsules can comprise components discussed elsewhere in this disclosure, such as a mushroom, fulvic acid, L-Theanine, Fish Oil, pregnenolone, phenyl ethyl amine (PEA), tulsi, lemon balm, passion flower, terpene compounds, blue lotus, cacao, maca, schizandra, Siberian ginseng, kava, skullcap, valerian, hops, California poppy, catuba, epidmedium, pao d'arco, ashwaganda, ginko, albiza, reishi, lion's mane, maitake, chaga, vitamin C, turmeric, cannabinoid compounds, cannabidiol (CBD), tetrahydrocannabinol (THC), bioperine, and xanthohumol oil-based compounds, and others, in microencapsulated form. In some cases, compositions can be encapsulated without the use of liposomes. In some cases, compositions can be encapsulated without the use of micelles. In some cases, compositions can be encapsulated without the use of liposomes or micelles. Compounds of the composition can exist within a microcapsule in forms including but not limited to liquid, gel, semi-solid, and solid. Microcapsules of compositions disclosed herein can further be processed into forms including but not limited to solids, powders, liquids, suspensions, gels, tablets, foods, lotions, cosmetics, and other forms discussed in this disclosure.

Microencapsulation can be performed with a microencapsulation device, including microfluidic droplet generation or encapsulation devices. An exemplary microencapsulation device is described, for example, in U.S. Pat. No. 7,482,152, incorporated here by reference in its entirety. Microfluidic droplets or emulsions can be generated by flow of a fluid to be encapsulated with an immiscible carrier fluid. For example, an oil fluid to be encapsulated can be flowed with an aqueous carrier fluid, or an aqueous fluid to be encapsulated can be flowed with an oil carrier fluid. Air can also be used as a fluid. Microfluidic droplet generators useful for microencapsulation include those employing co-flowing streams, cross-flowing streams (e.g., flow of streams at a T-junction), flow focusing, flow through perforated plates, and flow through nozzles. Droplet size can be controlled by parameters including device geometry, relative flow rates of the fluid streams, and operating pressure.

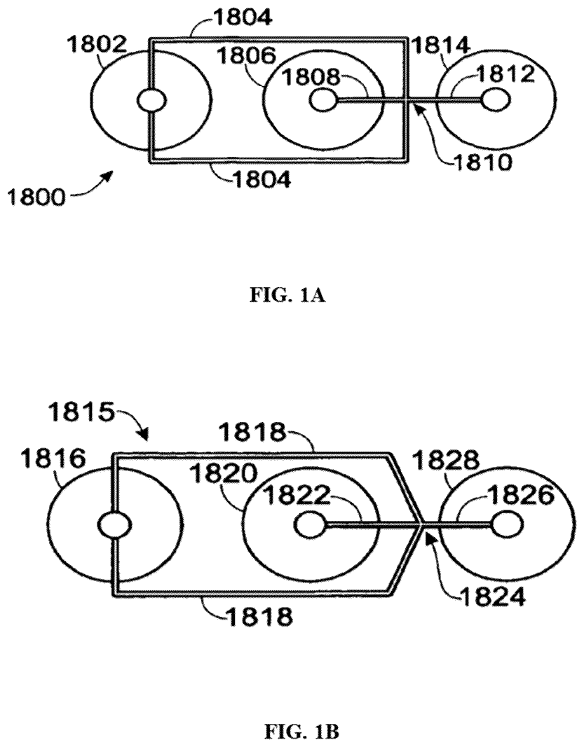

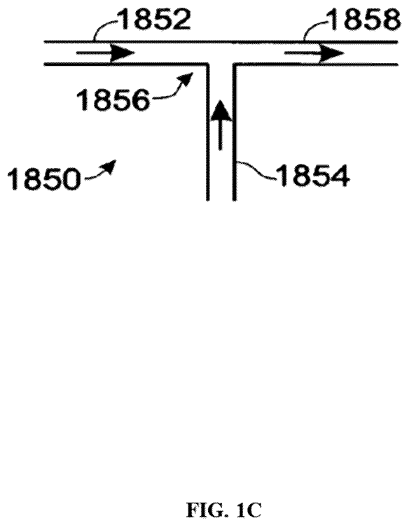

FIG. 1A shows an example of a droplet generator. In configuration 1800, a first phase (e.g., oil) from a first fluid chamber 1802 is transferred through two branches of a fluid channel section 1804. The first phase from the first fluid chamber 1802 intersects with a second phase (e.g., aqueous phase) from a second fluid chamber 1806, which is transferred along a fluid channel section 1808 to an intersection 1810 with the fluid channel section 1804. For example, the first phase from the first fluid chamber 1802 arrives at intersection 1810 from two different and substantially opposite directions, whereas the second phase arrives at the intersection along only a single path that is substantially perpendicular to both directions of travel of the arriving first phase fluid. At intersection 1810, droplets in the second phase are generated in a first phase background (e.g., a water-in-oil emulsion) and transferred along a fluid channel section 1812 to a third fluid chamber 1814, where the emulsion can be temporarily stored and/or transferred to another location. The phases can be reversed, for example, such that the droplets in the first phase are generated in a second phase background (e.g., an oil-in-water emulsion).

FIG. 1B shows another example of a droplet generator. In configuration 1815, a first phase (e.g., oil) from a first fluid chamber 1816 is transferred through two branches of a fluid channel section 1818. Fluid channel section 1818 intersects with a fluid channel section 1822 that transfers a second phase (e.g., aqueous) fluid from a second fluid chamber 1820, at intersection 1824. As in configuration 1800 of FIG. 1A, the first phase from the first fluid chamber 1816 arrives at intersection 1810 from two different directions, but unlike in configuration 1800, the fluid does not arrive from substantially opposite (antiparallel) directions. Rather, the branches of channel section 1818 each intersect channel section 1822 at a non-perpendicular angle, which is depicted as approximately 60 degrees in FIG. 1B. In general, configuration 1815 may include first phase fluid channels that intersect a second phase fluid channel at any desired angle or angles. The first phase fluid flowing through channel sections 1818 and the second phase fluid flowing through channel section 1822 can combine to form an emulsion of droplets in the second phase suspended in a first phase background (e.g., water-in-oil emulsion). As in the case of configuration 1800, the droplets then may be transferred along a fluid channel section 1826 to a third fluid chamber 1828, for storage and/or transfer to another location. The phases can be reversed, for example, such that the droplets in the first phase are generated in a second phase background (e.g., an oil-in-water emulsion).

FIG. 1C shows an example of a microfluidic structure. The arrows within the depicted fluid channels indicate the direction of fluid flow within each channel. Although fluid chambers for receiving and/or storing oil, water, and any generated emulsion are not depicted, these chambers or at least some source of oil and aqueous fluid may be present in a cartridge containing any of the depicted configurations. The fluid channels and any associated chambers may be formed by any suitable method, such as injection molding complementary sections of thermoplastic as described previously. In a T-junction configuration 1850, a first phase fluid traveling along channel 1852 intersects with a second phase fluid traveling along channel 1854 at intersection 1856, to produce second phase-in-first phase emulsions (e.g., water-in-oil, oi-in-water, etc.) that travels along outgoing fluid channel 1858. Droplets formed in the T-junction configuration 1850 may be formed primarily by a shear mechanism rather than primarily by a compression mechanism. However, droplet formation may depend on many factors, including the channel diameters, fluid velocities, and fluid viscosities.

Microencapsulation can be performed at a range of operating parameters, such as different flow rates or pressures. Microencapsulation can be conducted at a pressure of at least about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, 50000 psi, or more. Microencapsulation can be conducted at a pressure of at most about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, or 50000 psi. Microencapsulation can be conducted at a pressure of about 10 pounds per square inch (psi), 20 psi, 30 psi, 40 psi, 50 psi, 60 psi, 70 psi, 80 psi, 90 psi, 100 psi, 200 psi, 300 psi, 400 psi, 500 psi, 600 psi, 700 psi, 800 psi, 900 psi, 1000 psi, 2000 psi, 3000 psi, 4000 psi, 5000 psi, 6000 psi, 7000 psi, 8000 psi, 9000 psi, 10000 psi, 15000 psi, 20000 psi, 25000 psi, 30000 psi, 35000 psi, 40000 psi, 45000 psi, 50000 psi, or more. Microencapsulation can be conducted at a flow rate of at least about 1 milliliter per minute (mL/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 m/min, 60 mL/min, 70 mL/min, 80 mL/min, 90 mL/min, 100 mL/min, 110 mL/min, 120 m/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, 500 mL/min, or more. Microencapsulation can be conducted at a flow rate of at most about 1 milliliter per minute (mL/min), 2 mL/min 3 m/min, 4 mL/min, 5 mL/min, 6 mL/min, 7 mL/min, 8 mL/min, 9 mL/min, 10 mL/min, 20 mL/min, 30 mL/min, 40 mL/min, 50 mL/min, 60 mL/min, 70 mL/min, 80 mL/min, 90 mL/min, 100 mL/min, 110 mL/min, 120 mL/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, or 500 mL/min. Microencapsulation can be conducted at a flow rate of about 1 milliliter per minute (m/min), 2 mL/min 3 mL/min, 4 mL/min, 5 mL/min, 6 mL/min, 7 mL/min, 8 mL/min, 9 mL/min, 10 mL/min, 20 mL/min, 30 mL/min, 40 mL/min, 50 mL/min, 60 mL/min, 70 mL/min, 80 mL/min, 90 mL/min, 100 mL/min, 110 mL/min, 120 mL/min, 130 mL/min, 140 mL/min, 150 mL/min, 160 mL/min, 170 mL/min, 180 mL/min, 190 mL/min, 200 mL/min, 210 mL/min, 220 mL/min, 230 mL/min, 240 mL/min, 250 mL/min, 260 mL/min, 270 mL/min, 280 mL/min, 290 mL/min, 300 mL/min, 310 mL/min, 320 mL/min, 330 mL/min, 340 mL/min, 350 mL/min, 360 mL/min, 370 mL/min, 380 mL/min, 390 mL/min, 400 mL/min, 410 mL/min, 420 mL/min, 430 mL/min, 440 mL/min, 450 mL/min, 460 mL/min, 470 mL/min, 480 mL/min, 490 mL/min, 500 mL/min, or more.

Droplet generators can employ multiple parallel droplet generation operations in parallel. For example, a droplet generator (e.g., a plate, a device with channels) can employ at least 1, 2, 3, 4, 5, 6, 7, 8, 9, 10, 15, 20, 25, 30, 35, 40, 45, 50, 55, 60, 65, 70, 75, 80, 85, 90, 95, 100, or more droplet generating features (e.g., holes, channels, nozzles). A droplet generator can employ at most 1, 2, 3, 4, 5, 6, 7, 8, 9, 10, 15, 20, 25, 30, 35, 40, 45, 50, 55, 60, 65, 70, 75, 80, 85, 90, 95, or 100 droplet generating features. A droplet generator can employ about 1, 2, 3, 4, 5, 6, 7, 8, 9, 10, 15, 20, 25, 30, 35, 40, 45, 50, 55, 60, 65, 70, 75, 80, 85, 90, 95, 100, or more droplet generating features.

Microencapsulation can be performed via an emulsification process. For example, compositions can be emulsified in a mixer, such as an agitator, impeller, centrifugal mixer, or high-shear mixer. High-shear mixers can include batch high-shear mixers and inline high-shear mixers (e.g., rotor-stator mixers). Emulsification can also be conducted without a mixer, by combining fluids thermodynamically favored to form an emulsion, optionally with the aid of one or more emulsifiers or surfactants.

Microencapsulation processes can be conducted with the aid of one or more emulsifiers or surfactants. Emulsifiers and surfactants can include but are not limited to saponins (e.g., quillaja tree extract such as Q-NATURALE®, yucca extract), lecithin, soy lecithin, mustard seed hull extract, sodium stearoyl lactylate, polysorbate 20, and combinations thereof.

Microcapsules can comprise one or more stabilizers or gelling agents, which can be used to stabilize a microcapsule or emulsion. Stabilizers or gelling agents can include but are not limited to alginate (also algin or alginic acid) and agar. Alginate can be used in a variety of forms, including but not limited to inorganic salts such as sodium alginate, potassium alginate, calcium alginate, and combinations thereof. Alginate can be derived from sources such as seaweed (e.g., Macrocystis pyrifera, Ascophyllum nodosum, Laminaria spp.) or bacteria (e.g., Pseudomonas spp., Azotobacter spp.). Cross-linking agents or solutions, such as calcium chloride, can be used to stabilize or gel microcapsules.

Microcapsules can be characterized by a size (e.g., a diameter). The microcapsule size can be about 0.154 micrometers. The microcapsule size can be less than or equal to about 0.154 micrometers. The microcapsule size can be greater than or equal to about 0.154 micrometers. The microcapsule size can be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be less than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be greater than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The microcapsule size can be from about 0.1 to about 0.2 micrometers. The microcapsule size can be from about 0.05 to about 0.25 micrometers. The microcapsule size can be from about 0.05 to about 0.55 micrometers. The microcapsule size can be from about 0.05 to about 1 micrometers. The size distribution in a population of microcapsules can be homogeneous or substantially homogeneous. For example, a population of microcapsules can be characterized by dispersity, or polydispersity index (PDI), of less than or equal to about 20, 19, 18, 17, 16, 16, 15, 14, 13, 12, 11, 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.45, 1.40, 1.35, 1.30, 1.25, 1.20, 1.15, 1.14, 1.13, 1.12, 1.11, 1.10, 1.09, 1.08, 1.07, 1.06, 1.05, 1.04, 1.03, 1.02, 1.01, or 1.00.

### Solid Compositions and Delivery

The compositions of the present disclosure can comprise solid compositions (e.g., tablets, pills, etc.).

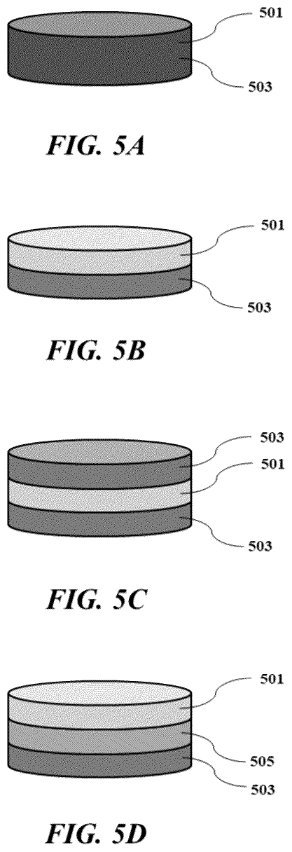

FIGS. 5A-5D show different examples of the solid composition (e.g., tablets) as disclosed herein. As shown in FIG. 5A, the tablet can comprise a mixture (e.g., homogeneous or heterogeneous mixture) of a non-cannabinoid therapeutic compound 501 and a cannabinoid compound 503. As shown in FIG. 5B, the tablet can comprise a first layer comprising a non-cannabinoid therapeutic compound 501 and a second layer comprising a cannabinoid compound 503. As shown in FIG. 5C, the tablet can comprise a middle layer comprising a non-cannabinoid therapeutic compound 501 that is disposed between at least two layers comprising a cannabinoid compound 503. As shown in FIG. 5D, the tablet can comprise a first layer comprising a non-cannabinoid therapeutic compound 501, a third layer comprising a cannabinoid compound 503, and a second layer comprising a filler or a different therapeutic compound 505 that is disposed between the first layer and the second layer.

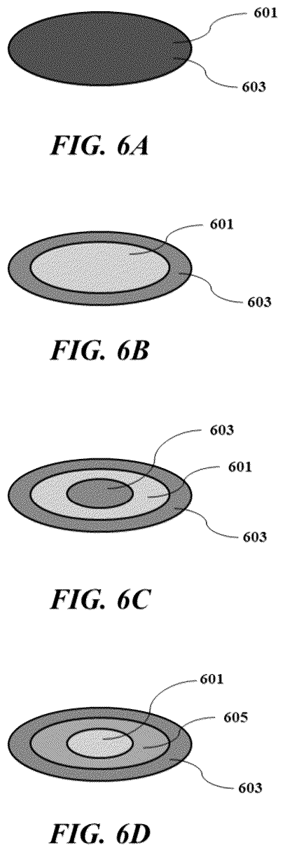

FIGS. 6A-6D show different examples of the solid composition (e.g., pills) as disclosed herein. As shown in FIG. 6A, the pill can comprise a mixture (e.g., homogeneous or heterogeneous mixture) of a non-cannabinoid therapeutic compound 601 and a cannabinoid compound 603. As shown in FIG. 6B, the pill can comprise a first layer comprising a non-cannabinoid therapeutic compound 601 and a second layer comprising a cannabinoid compound 603. As shown in FIG. 6C, the pill can comprise a middle layer comprising a non-cannabinoid therapeutic compound 601 that is disposed between at least two layers comprising a cannabinoid compound 603. As shown in FIG. 6D, the pill can comprise a first layer comprising a non-cannabinoid therapeutic compound 601, a third layer comprising a cannabinoid compound 603, and a second layer comprising a filler or a different therapeutic compound 605 that is disposed between the first layer and the second layer.

### Compositions

Therapeutic compositions of the present disclosure may comprise a variety of compounds, such as a mushroom, fulvic acid, L-Theanine, Fish Oil, pregnenolone, phenyl ethyl amine (PEA), tulsi, lemon balm, passion flower, terpene compounds, blue lotus, cacao, maca, schizandra, Siberian ginseng, kava, skullcap, valerian, hops, California poppy, catuba, epidmedium, pao d'arco, ashwaganda, ginko, albiza, reishi, lion's mane, maitake, chaga, vitamin C, turmeric, cannabinoid compounds, cannabidiol (CBD), tetrahydrocannabinol (THC), bioperine, xanthohumol oil-based compounds, and others, and/or a combination thereof. Cannabinoids utilized in the compositions disclosed herein include but are not limited to cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C₄ (CBN-C₄), cannabinol-C₂ (CBN-C₂), cannabiorcol (CBN-C₁), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), and cannabiripsol (CBR) cannabinoids.

Cannabinoids used in compositions of the present disclosure can be derived from various sources, including but not limited to hemp (e.g. hemp stalk, hemp stem, hemp seed), cannabis (e.g., cannabis flower, cannabis leaf, cannabis stalk, cannabis stem, cannabis seed), Echinacea purpurea, Echinacea angustifolia, Echinacea pallida, Acmella oleracea, Helichrysum umbraculigerum, Radula marginata, kava, black truffle, Syzygium aromaticum (cloves), Rosmarinus oficinalis, basil, oregano, black pepper, lavender, true cinnamon, malabathrum, Cananga odorata, copaifera spp., and hops.

Encapsulated cannabinoids can be present in a quantity of at least about 1, 2, 3, 4, 5, 6, 7, 8, 9, 10, 11, 12, 13, 14, 15, 16, 17, 18, 19, 20, 25, 30, 35, 40, 45, 50 nanograms, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of at most about 1, 2, 3, 4, 5, 6, 7, 8, 9, 10, 11, 12, 13, 14, 15, 16, 17, 18, 19, 20, 25, 30, 35, 40, 45, 50 nanograms, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of about 1, 2, 3, 4, 5, 6, 7, 8, 9, 10, 11, 12, 13, 14, 15, 16, 17, 18, 19, 20, 25, 30, 35, 40, 45, 50 nanograms, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabinoids can be present in a quantity of from about 1 to about 10 micrograms per microcapsule. Encapsulated cannabinoids can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabinoids can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabinoids can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Cannabinoids can be present in the composition (e.g., a product, such as a drug unit dose or a food product), in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in the composition (e.g., a product, such as a drug unit dose or a food product), in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in the composition (e.g., a product, such as a drug unit dose or a food product), in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in the composition (e.g., a product, such as a drug unit dose or a food product), in a quantity of from about 50 to about 150 milligrams. Cannabinoids can be present in the composition (e.g., a product, such as a drug unit dose or a food product), in a quantity of at least about 0.010%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present in the composition (e.g., a product, such as a drug unit dose or a food product), in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present in the composition (e.g., a product, such as a drug unit dose or a food product), in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The cannabinoids of the compositions disclosed herein can comprise cannabidiol-class compounds, including but not limited to cannabidiol (CBD), cannabidiolic acid (CBDA), cannabidiol monomethylether (CBDM), cannabidiol-C₄ (CBD-C₄), cannabidivarin (CBDV), cannabidivarinic acid (CBDVA), cannabidiorcol (CBD-C₁), and combinations thereof. CBD can comprise delta-1-cannabidiol, delta-2-cannabidiol, delta-3-cannabidiol, delta-3,7-cannabidiol, delta-4-cannabidiol, delta-5-cannabidiol, delta-6-cannabidiol, and combinations thereof.

Encapsulated cannabidiol compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated cannabidiol compounds can be present in a quantity of from about 1 to about 10 micrograms per microcapsule. Encapsulated cannabidiol compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%1, 3%, 14%, 15%, 16%, 17%, 18%, 19%, 20%, 25%, 30%, 35%, 40%, 45%, or 50% by weight of a microcapsule. Encapsulated cannabidiol compounds can be present in a quantity of at most about 0.1%, 0.2%, 0.3%, 0.4%, 0.5%, 0.6%, 0.7%, 0.8%, 0.9%1, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated cannabidiol compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Cannabidiol compounds can be present in the composition (e.g., a product, such as a drug unit dose or a food product), in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in the composition (e.g., a product, such as a drug unit dose or a food product), in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in the composition (e.g., a product, such as a drug unit dose or a food product), in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabidiol compounds can be present in the composition (e.g., a product, such as a drug unit dose or a food product), in a quantity of from about 50 to about 150 milligrams. Cannabidiol compounds can be present in the composition (e.g., a product, such as a drug unit dose or a food product), in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabidiol compounds can be present in the composition (e.g., a product, such as a drug unit dose or a food product), in a quantity of at most about 0.01%, 0.02%, 0.03%, 0.04%, 0.05%, 0.06%, 0.07%, 0.08%, 0.09%0, 0.1%, 0.2%, 0.3%, 0.4%, 0.5%0, 0.6%, 0.7%, 0.8%, 0.9%, 1%, 2%, 3%, 4%, 5%, 6%, 7%, 8%, 9%, 10%, 11%, 12%, 13%, 14%, 1%, 16%, 17%, 18%, 19%, 20%, 25%, 30%, 35%, 40%, 45%, or 50% by weight of the product. Cannabidiol compounds can be present in the composition (e.g., a product, such as a drug unit dose or a food product), in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The compositions of the present disclosure can comprise tetrahydrocannabinol (THC) as a type of cannabinoids. THC can comprise delta-9-THC, delta-8-THC, and combinations thereof. THC can comprise delta-6a,7-tetrahydrocannabinol, delta-7-tetrahydrocannabinol, delta-8-tetrahydrocannabinol, delta-9,11-tetrahydrocannabinol, delta-9-tetrahydrocannabinol, delta-10-tetrahydrocannabinol, delta-6a,10a-tetrahydrocannabinol, and combinations thereof. Delta-9-tetrahydrocannabinol can comprise stereoisomers including (6aR,10aR)-delta-9-tetrahydrocannabinol, (6aS,10aR)-delta-9-tetrahydrocannabinol, (6aS,10aS)-delta-9-tetrahydrocannabinol, (6aR,10aS)-delta-9-tetrahydrocannabinol, and combinations thereof.

In cases where the compositions comprise microcapsules, THC compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of from about 1 to about 10 micrograms per microcapsule. Encapsulated THC compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated THC compounds can be present in a quantity of at least about 0.1%, 0.2%, 0.3%, 0.4%, 0.5%, 0.6%, 0.7%, 0.8%, 0.9%1, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated THC compounds can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated THC compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

THC compounds can be present in the composition (e.g., a product, such as a drug unit dose or a food product), in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). THC compounds can be present in the composition (e.g., a product, such as a drug unit dose or a food product), in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). THC compounds can be present in the composition (e.g., a product, such as a drug unit dose or a food product), in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). THC compounds can be present in the composition (e.g., a product, such as a drug unit dose or a food product), in a quantity of from about 50 to about 150 milligrams. THC compounds can be present in the composition (e.g., a product, such as a drug unit dose or a food product), in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. THC compounds can be present in the composition (e.g., a product, such as a drug unit dose or a food product), in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. THC compounds can be present in the composition (e.g., a product, such as a drug unit dose or a food product), in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

In some cases, a composition of the present disclosure does not contain a psychoactive amount of THC. For example, cannabinoids in compositions of the present disclosure can contain less than 100%, 90%, 80%, 70%, 60%, 50%, 40%, 30%, 20%, 10%, 5%, 1%, 0.7%, 0.5%, 0.3%, or 0.1% THC relative to the total quantity of cannabinoid compounds. In some cases, the ratio of a non-THC cannabinoid (e.g., cannabidiol) to THC in a composition of the present disclosure is greater than or equal to about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or 100:1. In some cases, compositions of the present disclosure contain less than 0.3% THC.

The compositions of the present disclosure can comprise one or more terpene compounds, including but not limited to terpenoids such as monoterpenoids, sesquiterpenoids, diterpenoids, and triterpenoids. Terpenes can be acyclic, monocyclic, or polycyclic. Terpenes can include but are not limited to myrcene, limonene, linalool, trans-ocimene, cis-ocimene, alpha-pinene, beta-pinene, alpha-humulene (alpha-caryophyllene), beta-caryophyllene, delta-3-carene, trans-gamma-bisabolene, cis-gamma-bisabolene, trans-alpha-famesene, cis-beta-famesene, beta-fenchol, beta-phellandrene, guajol, alpha-gualene, alpha-eudesmol, beta-eudesmol, gamma-eudesmol, terpinolene, alpha-selinene, beta-selinene, alpha-terpineol, fenchone, camphene, cis-sabinene hydrate, alpha-trans-bergamotene, alpha-cis-bergamotene, bomeol, gamma-curcumene, alpha-thujene, epi-alpha-bisabolol, ipsdienol, alpha-ylangene, beta-elemene, gamma-muurolene, alpha-cadinene, alpha-longipinene, caryophyllene oxide, and combinations thereof.

Encapsulated terpenes can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpenes can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpenes can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated terpene compounds can be present in a quantity of from about 1 to about 10 micrograms per microcapsule. Encapsulated terpenes can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 1%, 16%, 17%, 18%, 19%, 20%, 25%, 30%, 35%, 40%, 45%, or 50% by weight of a microcapsule. Encapsulated terpenes can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule. Encapsulated terpenes can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

Terpene compounds can be present in the composition (e.g., a product, such as a drug unit dose or a food product), in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in the composition (e.g., a product, such as a drug unit dose or a food product), in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in the composition (e.g., a product, such as a drug unit dose or a food product), in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in the composition (e.g., a product, such as a drug unit dose or a food product), in a quantity of from about 50 to about 150 milligrams. Terpene compounds can be present in the composition (e.g., a product, such as a drug unit dose or a food product), in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be present in the composition (e.g., a product, such as a drug unit dose or a food product), in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be present in the composition (e.g., a product, such as a drug unit dose or a food product), in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 400, %5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

Non-cannabinoid therapeutic compounds can be present in the composition (e.g., a product, such as a drug unit dose or a food product), in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Non-cannabinoid therapeutic compounds can be present in the composition (e.g., a product, such as a drug unit dose or a food product), in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Non-cannabinoid therapeutic compounds can be present in the composition (e.g., a product, such as a drug unit dose or a food product), in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Non-cannabinoid therapeutic compounds can be present in the composition (e.g., a product, such as a drug unit dose or a food product), in a quantity of from about 50 to about 150 milligrams. Non-cannabinoid therapeutic compounds can be present in the composition (e.g., a product, such as a drug unit dose or a food product), in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Non-cannabinoid therapeutic compounds can be present in the composition (e.g., a product, such as a drug unit dose or a food product), in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Non-cannabinoid therapeutic compounds can be present in the composition (e.g., a product, such as a drug unit dose or a food product), in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, %19%, 20%, 25%, 30%, 35%, 40%, 45%, or 50% by weight of the product.

For a composition as disclosed herein, an amount of a cannabinoid compound can be greater than an amount of a non-cannabinoid therapeutic compound by at least about 0.010%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1,000%, 2,000%, or more by weight. For a composition as disclosed herein, an amount of a cannabinoid compound can be greater than an amount of a non-cannabinoid therapeutic compound by at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 1%, 2%, %13%, %14%, 15%, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1,000%, 2,000%, or more by weight.

For a composition as disclosed herein, an amount of a cannabinoid compound can be less than an amount of a non-cannabinoid therapeutic compound by at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 2%, 3%, %14%, %15%, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1,000%, 2,000%, or more by weight. For a composition as disclosed herein, an amount of a cannabinoid compound can be less than an amount of a non-cannabinoid therapeutic compound by at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 3%, 4%, %15, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1,000%, 2,000%, or more by weight.

In some cases, an amount of a cannabinoid compound can be approximately the same as an amount of a non-cannabinoid therapeutic compound in the composition as disclosed herein.

Encapsulated non-cannabinoid therapeutic compounds can be present in a quantity of at least about 1, 2, 3, 4, 5, 6, 7, 8, 9, 10, 11, 12, 13, 14, 15, 16, 17, 18, 19, 20, 25, 30, 35, 40, 45, 50 nanograms, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated non-cannabinoid therapeutic compounds can be present in a quantity of at most about 1, 2, 3, 4, 5, 6, 7, 8, 9, 10, 11, 12, 13, 14, 15, 16, 17, 18, 19, 20, 25, 30, 35, 40, 45, 50 nanograms, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated non-cannabinoid therapeutic compounds can be present in a quantity of about 1, 2, 3, 4, 5, 6, 7, 8, 9, 10, 11, 12, 13, 14, 15, 16, 17, 18, 19, 20, 25, 30, 35, 40, 45, 50 nanograms, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per microcapsule. Encapsulated non-cannabinoid therapeutic compounds can be present in a quantity of from about 1 to about 10 micrograms per microcapsule. Encapsulated non-cannabinoid therapeutic compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%1, 3%1, 4%1, 5%1, 6%1, 7%1, 8%, 19%, 20%, 25%, 30%, 35%, 40%, 45%, or 50% by weight of a microcapsule. Encapsulated non-cannabinoid therapeutic compounds can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, %17%, 18%, 19%, 20%, 25%, 30%, 35%, 40%, 45%, or 50% by weight of a microcapsule. Encapsulated non-cannabinoid therapeutic compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a microcapsule.

The compositions of the present disclosure can be enriched in cannabinoids compared to hemp oil. For example, a composition can comprise hemp oil and cannabinoids from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of cannabinoids compared to hemp oil.

The compositions of the present disclosure can be enriched in cannabidiol compounds compared to hemp oil. For example, a composition can comprise hemp oil and cannabidiol compounds from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of cannabidiol compounds compared to hemp oil.

The compositions of the present disclosure can be enriched in THC compounds compared to hemp oil. For example, a composition can comprise hemp oil and THC compounds from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of THC compounds compared to hemp oil.

The compositions of the present disclosure can be enriched in terpenes compared to hemp oil. For example, a composition can comprise hemp oil and terpenes from plant sources such as extracts (e.g., hemp extract) and essential oils. A composition can comprise about 0%, 10%, 20%, 30%, 40%, 50%, 60%, 70%, 80%, 90%, 100%, 200%, 300%, 400%, 500%, 600%, 700%, 800%, 900%, or 1000% greater concentration of terpenes compared to hemp oil.

Compounds included in the compositions of the present disclosure can be derived from various sources. Compound sources can be natural, such as plant extracts or essential oils. Compounds in the compositions of the present disclosure can be derived from hemp oil, including cannabinoid compounds, THC compounds, and terpene compounds. Compounds in the compositions of the present disclosure can be derived from essential oils, including but not limited to those essential oils discussed further in this disclosure. These compounds can include cannabinoid compounds and terpene compounds. In some cases, all the compounds or ingredients in a composition are natural or naturally-derived. In some cases, all the compounds or ingredients in a composition are vegetarian. In some cases, all the compounds or ingredients in a composition are vegan.

Terpenes and/or essential oils in compositions of the present disclosure can be selected to provide benefits for particular conditions or subjects. Terpenes and/or essential oils can be employed in combination with each other, as well as in combination with cannabinoids, for example to target treatment of particular conditions. For example, terpinolene, terpineol and linalool or lavender, valerian and jasmine essential oils can be combined with cannabinoids or cannabis extract to act as a sleep aid or treat sleep disorders.

Alpha-pinene can be used as an anti-inflammatory, an antiangiogenic, an anti-ulcer agent, and a bronchodilator.

Linalool can be used for reducing anxiety, reducing inflammation (e.g., lung inflammation), to improve Alzheimer's disease or symptoms thereof, as a sedative, an analgesic, an anti-microbial, an antibacterial, and an anti-epileptic.

Myrcene can be used as an antibacterial, a neuroprotective agent, an antinociceptive, an analgesic, and to alleviate neuropathic pain, peptic ulcer disease, and inflammation. Depending on concentration, myrcene can be used as a sedative (e.g., over 0.5% myrcene) or to provide energizing effects (e.g., less than 0.5% myrcene).

Limonene can be used to reduce anxiety and depression, to dissolve cholesterol-containing gallstones, to neutralize gastric acid, support normal peristalsis, relieve heartburn and gastroesophageal reflux, to improve immune function, and as a chemopreventative against cancer.

Ocimene can be used as an antifungal agent, an antitumor agent, and a cyctotoxic agent.

Terpinolene can be used for antioxidant, mood regulation, central nervous system (CNS) regulation, anti-inflammatory, anti-diarrheal, anti-filarial, anti-fungal, antimalarial, anti-amoebic, anti-bacterial, cytotoxic, and anticancer effects.

Terpineol can be used to relax a subject, to aid digestion and improve gastrointestinal disorders, and to relieve influenza, bronchitis, cough, nasal congestion, and sinusitis.

Beta-caryophyllene can be used as an anti-inflammatory agent, an anti-tumor agent, and an analgesic.

Geraniol can be used to reduce or protect against neuropathy, as an antidepressant, to suppress angiogenesis, to improve anti-cancer agent efficacy, to suppress growth of cancer cells (e.g., lung cancer), as a chemopreventive against cancer, to reduce inflammation and apoptosis (e.g., in liver cells), to reduce oxidative stress, as an antioxidant, and as an antimicrobial.

Alpha-humulene can be used as an appetite suppressant, an anti-inflammatory agent, an insect repellant, an antibacterial, an antioxidant, and an allelopathic agent.

Phellandrene can be used as an antidepressant and an antihyperalgesic.

Carene can be used as an antioxidant, an antiproliferative, an antimicrobial, and to reduce excess body fluid production, such as of tears, mucous, or sweat.

Terpinene can be used as an antioxidant, an anti-inflammatory, an antimicrobial, an antiproliferative, to reduce oxidative stress, and to manage diabetes.

Fenchol can be used as an antibacterial agent, an antimycobacterial, an antimicrobial, and an antioxidant.

Borneol can be used to alleviate hyperalgesia, as a TRPA1 inhibitor, an anti-inflammatory agent, and an anti-nociceptive agent.

Bisabolol can be used as an anti-cancer agent, such as to induce apoptosis in leukemia, an anti-tumor agent (e.g., pancreatic cancer), and an antigenotoxicity agent.

Phytol can be used to relax a subject, such as by inhibiting degradation of GABA, as an anxiolytic, to resist menadione-induced oxidative stress, and as an antimicrobial.

Camphene can be used for pain relief, as an antioxidant, to induce apoptosis in cancer cells (e.g., melanoma), an antitumor agent, and an antibacterial.

Sabinene can be used as an antioxidant, an antimicrobial, an anticancer agent (e.g., oral, liver, lung, colon, melanoma, and leukemic cancer), to aid liver function, aid digestion, relieve arthritis, and relieve skin conditions.

Camphor can be used to improve skin healing (e.g., reconstructed human epidermis), as a local anesthetic, a muscle relaxant, an antipathogenic, and an antimicrobial agent.

Isobomeol can be used as an antioxidant, a cytotoxic, a DNA-protective, to inhibit herpes simplex virus type 1, and to inhibit HIV.

Menthol can be used as an analgesic, to desensitize α3β4 nicotinic acetylcholine receptors, as an antinociceptive, and as an anti-inflammatory agent.

Nerolidol can be used as an antifungal agent, an antimicrobial agent, an antioxidant, and an antimalarial agent.

Guaiol can be used as an antimicrobial agent, an antifungal agent, and an antibiotic.

Isopulegol can be used as a gastroprotective agent, an anti-inflammatory agent, to enhance permeability for transdermal administration of compounds, and to reduce the severity of seizures.

Geranyl acetate can be used as an antimicrobial agent, an antibacterial, and an antioxidant.

Cymene can be used as an anti-inflammatory agent, an anti-hyperalgesic, an antioxidant, an anti-diabetic, to aid in weight loss, to aid immune disorders, and to protect against acute lung injury.

Eucalyptol can be used as an antifungal agent, to alleviate inflammation (e.g., lung inflammation), an antioxidant, and an anticancer agent.

Pulegone can be used to enhance skin permeability, as an insecticide, and an antioxidant.

The compositions of the present disclosure can comprise one or more essential oils or essential oil compounds. Essential oils can include, but are not limited to: Linalool; B-Caryophyllene; B-Myrcene; D-Limonene; Humulene; a-Pinene; Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.); Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evemia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichrysum (Helichrysum italicum); Grapefruit, White (Citrus x paradisi); Grapefruit, Pink (Citrus paradisi); Ginger (Zingiber officinalis); Geranium (Pelargonium graveolens); Geranium, Bourbon (Pelargonium graveolens, ‘Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferus L.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and combinations thereof.

The compositions of the present disclosure can comprise one or more additional ingredients, including but not limited to mushrooms or mushroom derivative products (e.g., reishi mushroom, chaga mushroom, maitake mushroom, oyster mushroom, cordyceps), maca (Lepidium meyenii), he sho wu (also he show wu or shou wu chih), superfoods or superfood derivative products (e.g., blueberries, acai berries, inca berries, goji berries, camucamu, coconut, lucuma, kale, cacao (e.g., cacao powder, cacao butter), sacha inchi, chia, flax, hemp, amaranth, quinoa, moringa oleifera), and combinations thereof.

Compounds used in compositions of the present disclosure can be extracted by a variety of methods. For example, extraction can be performed by maceration, infusion, decoction, percolation, Soxhlet extraction, pressurized solvent extraction, counter current extraction, ultrasonication, or supercritical fluid (e.g., carbon dioxide) extraction.

In some cases, compounds used in compositions of the present disclosure are extracted via supercritical fluid (e.g., carbon dioxide) extraction. For example, cannabinoid compounds can be extracted from hemp (e.g., hemp stalk and hemp stems) using supercritical carbon dioxide extraction.

The compositions of the present disclosure can comprise pregnenolone, including derivatives thereof. Pregnenolone can help protect a subject from cannabis intoxication, for example from THC. Pregnenolone or derivatives thereof can be formulated to be water soluble. A composition of the present disclosure can comprise between about 1 and 50 milligrams (mg) of pregnenolone or derivatives thereof. For example, a unit dosage of the present disclosure can comprise between about 1 and 50 milligrams (mg) of pregnenolone. Compositions of the present disclosure (e.g., unit dosages) can comprise about 1, 2, 3, 4, 5, 6, 7, 8, 9, 10, 15, 20, 25, 30, 35, 40, 45, or 50 mg of pregnenolone. Compositions of the present disclosure (e.g., unit dosages) can comprise at least about 1, 2, 3, 4, 5, 6, 7, 8, 9, 10, 15, 20, 25, 30, 35, 40, 45, or 50 mg of pregnenolone. Compositions of the present disclosure (e.g., unit dosages) can comprise at most about 1, 2, 3, 4, 5, 6, 7, 8, 9, 10, 15, 20, 25, 30, 35, 40, 45, or 50 mg of pregnenolone. Compositions comprising pregnenolone can be used in combination with any other compounds, ingredients, or formulations described herein, including esters, cyclodextrin complexes, microcapsules (e.g., sodium alginate microcapsules), immediate release formulations, delayed or extended release formulations, transbuccal formulations, and sublingual formulations.

Compositions of the present disclosure can be used to treat various diseases or conditions in subjects (e.g., humans, mammals, vertebrates), including but not limited to ALS, Alzheimer's, antibacterial resistant infections, anxiety, atherosclerosis, arthritis, asthma, cancer, colitis, Crohn's, diabetes, depression, endocrine disorders, epilepsy, seizures, fibromyalgia, glaucoma, heart disease, Huntington's, inflammation, irritable bowel syndrome (IBS), kidney disease, liver disease, motion sickness, nausea, neurodegeneration, neuropathic pain, neuropathy, obesity, obsessive compulsive disorder (OCD), osteoporosis, Parkinson's, prion diseases, Mad Cow disease, post-traumatic stress disorder (PTSD), rheumatism, schizophrenia, sickle cell anemia, skin conditions (e.g., psoriasis, dermatitis, allergic inflammation, chronic pruritus), sleep disorders (e.g., sleep-wake disorders, apnea), spinal cord injury, stress, stroke, and traumatic brain injury (TBI). The compositions of the present disclosure can be provided as a dry powder. For example, an oil-based composition (e.g., hemp oil) can be combined with a drying or powdering agent, such as cyclodextrin. In some cases, a powder composition can be provided on its own. In other cases, a powder composition can be provided in another product, such as a food product, cosmetic product, or other products and compositions such as those disclosed herein.

The compositions of the present disclosure can be provided in any suitable form, including but not limited to a liquid form, a gel form, a semi-liquid (e.g., a liquid, such as a viscous liquid, containing some solid) form, a semi-solid (a solid containing some liquid) form, or a solid form. Compositions can be provided in, for example, a tablet form, a capsule form, a food form a chewable form, a non-chewable form, a transbuccal form, a sublingual form, a slow-release form, a non-slow-release form, a sustained release form, or a non-sustained-release form.

The compositions of the present disclosure can be administered in any oral dosage form, including liquid dosage forms (e.g., a suspension or slurry), and oral solid dosage forms (e.g., a tablet or bulk powder). Tablets can include tablets, caplets, capsules, including soft gelatin capsules, and lozenges. Tablets can further comprise suitable binders, lubricants, diluents, disintegrating agents, colorants, flavoring agents, flow-inducing agents, and melting agents.

The compositions of the present disclosure can be administered transdermally, such as via a patch. The compositions of the present disclosure can be administered intravenously. The compositions of the present disclosure can be administered topically. The compositions of the present disclosure can be administered via exposure to an aqueous solution, such as a subject immersing in a float tank. The compositions of the present disclosure can be formulated as a bath salt or liquid bath product, which can be dissolved or dispersed in water (e.g., a bath) for skin exposure, for example by immersion of the subject.

The compositions of the present disclosure can be provided as cosmetics or personal care products, such as soaps (e.g., solid, bar, liquid, or foaming), hand sanitizer, lotions, massage oils masks, makeup, moisturizers, sunscreen, toothpaste, mouth wash, or throat spray. Use of cannabinoids in such applications can provide benefits including reduction of inflammation in a subject.

The compositions of the present disclosure can be provided as a food composition in combination with a food carrier, including but not limited to food bars (e.g., granola bars, protein bars, candy bars), cereal products (e.g., oatmeal, breakfast cereals, granola), bakery products (e.g., bread, donuts, crackers, bagels, pastries, cakes), dairy products (e.g., milk, yogurt, cheese), beverages (e.g., milk-based beverages, sports drinks, fruit juices, teas, soft drinks, alcoholic beverages, bottled waters), beverage mixes, pastas, grains (e.g., rice, corn, oats, rye, wheat, flour), egg products, snacks (e.g., candy, chips, gum, gummies, lozenges, mints, chocolate), meats, fruits, vegetables or combinations thereof. Food compositions can comprise solid foods. Food compositions can comprise semi-solid foods. Food compositions can comprise liquid foods. A composition in a liquid form may be formulated from a dry mix, such as a dry beverage mix or a powder. A dry mix may be suitable in terms of transportation, storage, or shelf life. The composition can be formulated from the dry mix in any suitable manner, such as by adding a suitable liquid (e.g., water, milk, fruit juice, tea, or alcohol).

A food composition or food product can comprise a food bar, including but not limited to granola bars, protein bars, candy bars, and energy bars. A food composition or food product can comprise a cereal product, including but not limited to oatmeal, flour (e.g., wheat flour, rice flour, corn flour, barley flour), breakfast cereal, granola, bread, pasta, rice cakes, and popcorn. A food composition or food product can comprise a bakery product, including but not limited to bread, pastries, brownies, cakes, pies, donuts, crackers, and muffins. A food composition or food product can comprise a dairy product, including but not limited to milk, fermented milk, curd, whey, yogurt, cream, cheese, butter, clarified butter, ghee, and ice cream. A food composition or food product can comprise a nut butter or seed butter, including but not limited to peanut butter, almond butter, cashew butter, hazelnut butter, macadamia nut butter, pecan butter, pistachio butter, walnut butter, pumpkin seed butter, sesame seed butter, soybean butter, and sunflower seed butter. A food composition or food product can comprise an oil (e.g., a cooking oil), including but not limited to olive oil, coconut oil, vegetable oil, canola oil, corn oil, peanut oil, sunflower seed oil, almond oil, avocado oil, rice bran oil, cottonseed oil, flaxseed oil, linseed oil, grape seed oil, hemp oil, mustard oil, macadamia oil, palm oil, tea seed oil, walnut oil, margarine, lard, butter, clarified butter, ghee, or tallow. A food composition or food product can comprise sports food products such as energy gels, sports drinks, energy powders, energy bars, energy shots, protein powders, and protein drinks (e.g., protein shakes). A food composition or food product can comprise a beverage, including but not limited to water, electrolyte drinks, soda, coconut water, tea (e.g., Jun tea, black tea, green tea, white tea, herbal tea), coffee, a soft drink, an alcoholic beverage (e.g., cocktail, liquor, spirits, beer, wine, malt beverage), water, juice (e.g., apple juice, orange juice, tomato juice, vegetable juice, cranberry juice), a sports drink, electrolyte-enriched water, vitamin-enhanced water, a hangover-recovery drink, milk (e.g., dairy-based milk, coconut milk, almond milk, soy milk, hemp milk, rice milk, oat milk, cashew milk, hazelnut milk), and yogurt. A food composition or food product can comprise a fungus or fermented food or drink, including but not limited to kifir (kefir), jun, amasi, amazake, appam, ayran, doogh, bagoong, brem, cheonggukjang, chicha, kombucha, fermented bean curd, kimchi, lassi, miso, poi, yakult, and yogurt.

Compositions of the present disclosure can comprise pet or other animal products, such as animal food (e.g., dog food, cat food), treats, and nutritional supplements (e.g., liquids, sprays, or powders for application to food or water). These compositions can be formulated for or administered to domestic or pet animals (e.g., dogs, cats, small mammals, birds), livestock and other farm animals (e.g., cows, pigs, horses, sheep, goats), zoo animals, or any other vertebrates. Compositions for administration to animals can be formulated with microencapsulated cannabinoid-rich oil or non-encapsulated cannabinoid-rich oil, alone or in combination with essential oils, terpenes, and other components described herein. Compositions for administration to animals can be mixed into feed or water, prepared for spraying application (e.g., mixed in glycerin), for intravenous administration (e.g., in a syringe or an IV bag), in salves, vitamins, liquid vitamin pumps, treats, or other forms.

The compositions of the present disclosure can comprise an additional agent or agents, whether active or passive. Examples of such an agent include a sweetening agent, a flavoring agent, a coloring agent, a filling agent, a binding agent, a lubricating agent, an excipient, a preservative, or a manufacturing agent. Additional pharmaceutically acceptable excipients (in the case of pharmaceuticals) or other additives (for non-pharmaceutical applications) can be added to the composition. For example, if desired, any generally accepted soluble or insoluble inert pharmaceutical filler (diluent) material can be included in the final product (e.g., a solid dosage form). Such inert pharmaceutical filler can comprise a monosaccharide, a disaccharide, a polyhydric alcohol, inorganic phosphates, sulfates or carbonates, and combinations thereof. Examples of suitable inert pharmaceutical fillers include sucrose, dextrose, lactose, xylitol, fructose, sorbitol, calcium phosphate, calcium sulfate, calcium carbonate, microcrystalline cellulose, and combinations thereof. An effective amount of any generally accepted pharmaceutical lubricant, such as calcium or magnesium soaps, can be added.

The compositions of the present disclosure can be administered to a subject. Compositions can be administered in a variety of ways, including but not limited to oral and topical administration.

Administering the compositions of the present disclosure to a subject can provide one or more beneficial effects. Beneficial effects can include but are not limited to pain relief, reduced bacterial growth, reduced blood sugar levels, improved blood lipid and cholesterol profiles, increased fat burning, reduced appetite, stimulated appetite, reduced vomiting or nausea, reduced seizures or convulsions, antifungal effects, reduced inflammation, reduced arthritis (e.g., rheumatoid arthritis), reduced insomnia or aided sleep, reduced arterial blockage, inhibited cancer cell growth, improved psoriasis, tranquilizing effects, antispasmodic effects, reduced anxiety, bone growth promotion, reduced intestinal contractions, and nervous system protection.

Administering a composition of the present disclosure to a subject can improve a therapeutic window of the subject. The improved therapeutic window may be compared to administration of the non-cannabinoid therapeutic compound individually. Administering a composition of the present disclosure can mitigate a toxicity of the non-cannabinoid therapeutic compound, as compared to administering the non-cannabinoid therapeutic compound individually.

Any of the subject compositions can be provided in a unit dosage form. A unit dosage is an amount of a compound, such as a cannabinoid compound delivered with other components (e.g., non-cannabinoid therapeutic compounds as disclosed herein), which is to be administered to a subject (e.g., orally, intravenously, etc.) at or about one time point. Other components which can be included with a unit dosage include but are not limited to cosmetics, food carriers, food bars, baked goods, dairy products, oils, beverages, solid dosages (e.g., tablets or pills), or liquid dosages. A unit dosage of a cannabinoid compound can be about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a non-cannabinoid therapeutic compound can be about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a non-cannabinoid therapeutic compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a non-cannabinoid therapeutic compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage can be an hourly dosage. A unit dosage can be a daily dosage. A unit dosage can provide about 1/24, 1/12, ⅛, ⅙, ¼, ⅓, ½, or all of a daily dosage of one or more cannabinoids or at least one non-cannabinoid therapeutic compounds for a subject. A unit dosage can take the form of a tablet, gel, liquid, food product, food bar, container of liquid of defined volume, or other forms described herein, packaged for one-time consumption or administration.

The compositions described herein can provide several advantages, including but not limited to increased shelf stability, increased bioavailability, increased bioactivity, and delayed release. The compositions described herein, when administered to a subject, can have various release profiles, half-lives, and metabolic characteristics. The subject compositions can comprise a plurality of microcapsules, wherein an individual microcapsule in the plurality is characterized by exhibiting at least one of: (a) a sigmoidal release profile of the at least one cannabinoid compound; (b) a plasma half-life of the at least one cannabinoid compound greater than twice that of the at least one cannabinoid compound in non-encapsulated form; (c) a first pass metabolism of the at least one cannabinoid compound reduced by at least 50% compared to the at least one cannabinoid compound in non-encapsulated form; d) a rate of excretion of the at least one cannabinoid compound from a subject's body reduced by at least 20% compared to the at least one cannabinoid compound in non-encapsulated form; or (e) a degradation rate at an ambient temperature of at least 20° C. of the at least one cannabinoid compound of less than about 50% of a degradation rate of the at least one cannabinoid compound in non-encapsulated form.

The compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years. Compositions in microencapsulated form can be characterized by a cannabinoid degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non-encapsulated cannabinoid composition.

Cannabinoid compositions in microencapsulated form can be characterized by a plasma half-life in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, 15, 16, 17, 18, 19, 20, 25, 30, 35, 40, 45, 50, 60 70, 80, 90, or 100 times that of a non-encapsulated cannabinoid composition. Plasma half-life of a composition can be determined experimentally by administering the composition to a subject, taking plasma samples from a subject at multiple time points, and measuring the concentration of the compound or compounds of interest in those plasma samples. The concentration of the compound or compounds of interest will reach a peak value in the plasma, then fall as the compound or compounds are metabolized, degraded, or cleared from the blood stream. The plasma half-life is the time for the plasma concentration value to be halved.

The cannabinoid release profile can be sigmoidal (e.g., having an ‘S’ shape curve, such as a logistic function). The cannabinoid release profile can be non-sigmoidal. The cannabinoid release profile can be linear. The cannabinoid release profile can be non-linear. The cannabinoid release profile can be instant release. The cannabinoid release profile can be non-instant release. The cannabinoid release profile can be delayed release. The cannabinoid release profile can be constant or sustained release. The cannabinoid release profile can be non-constant or non-sustained release.

Tablets can be formulated in sustained release format. There are various methods of making sustained release tablets; see, for example, U.S. Patent Publication No. 2006/0051416 and U.S. Patent Publication No. 2007/0065512, each of which is entirely incorporated herein by reference. Examples of gradual-release tablets are provided in U.S. Pat. No. 3,456,049, which is entirely incorporated herein by reference. A slow- or sustained-release form may delay disintegration or absorption of the composition or one or more components thereof.

In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 1 hour of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 2 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 3 hours of administration to a subject. In some cases, no more than 5%, 10%, 15%, 20%, 25%, 30%, 35%, 40%, 45%, 50%, 55%, 60%, 6%, 70, 7%, 80, 85%, 90%, 95%, 96%, 97%, 98%, or 99% of a cannabinoid compound is released from a microcapsule within 4 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 5 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 6 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 7 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a cannabinoid compound is released from a microcapsule within 8 hours of administration to a subject.

A release profile is the relationship between time and the amount of a compound released into a subject or the concentration of the compound within the subject (e.g., within the plasma). Release profiles can be measured in a similar manner to plasma half-life. A composition can be administered to a subject, and samples (e.g., plasma samples or blood samples) can be taken from the subject at multiple time points. The concentration of the compound or compounds of interest can be measured in those samples, and a release profile can be plotted.

Compounds taken up into a subject via the gastrointestinal system can be transported to the liver before entering general circulation. Compounds susceptible to metabolic degradation in the liver can have their activities substantially reduced by the first-pass metabolism through the liver. Encapsulation (e.g., microencapsulation) of compounds can reduce first-pass metabolism of the compounds in the liver. Compositions in microencapsulated form can be characterized by a first pass cannabinoid metabolism in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a non-encapsulated cannabinoid composition. Compositions in microencapsulated form can be characterized by a cannabinoid excretion rate from a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a non-encapsulated cannabinoid composition.

The compositions described herein, when administered to a subject, can have improved bioavailability, bioactivity, or both. Bioavailability is the fraction of an administered dosage of unchanged compound that reaches systemic circulation. Cannabinoid compositions in microencapsulated form can be characterized by a bioavailability in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of a non-encapsulated cannabinoid composition. Cannabinoid compositions in microencapsulated form can be characterized by a bioavailability in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%,%, 98%, 99%, or 100%.

Bioactivity, or biological activity, is the activity exerted by the active ingredient or ingredients in a composition. Cannabinoid compositions in microencapsulated form can be characterized by a bioactivity in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of a non-encapsulated cannabinoid composition.

The subject compositions can comprise a plurality of microcapsules, wherein an individual microcapsule in the plurality is characterized by exhibiting at least one of: (a) a sigmoidal release profile of the at least one non-cannabinoid therapeutic compound; (b) a plasma half-life of the at least one non-cannabinoid therapeutic compound greater than twice that of the at least one non-cannabinoid therapeutic compound in non-encapsulated form; (c) a first pass metabolism of the at least one non-cannabinoid therapeutic compound reduced by at least 50% compared to the at least one non-cannabinoid therapeutic compound in non-encapsulated form; d) a rate of excretion of the at least one non-cannabinoid therapeutic compound from a subject's body reduced by at least 20% compared to the at least one non-cannabinoid therapeutic compound in non-encapsulated form; or (e) a degradation rate at an ambient temperature of at least 20° C. of the at least one non-cannabinoid therapeutic compound of less than about 50% of a degradation rate of the at least one non-cannabinoid therapeutic compound in non-encapsulated form.

The compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years. Compositions in microencapsulated form can be characterized by a non-cannabinoid therapeutic compound degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non-encapsulated non-cannabinoid therapeutic compound.

Non-cannabinoid therapeutic compounds in microencapsulated form can be characterized by a plasma half-life in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, 15, 16, 17, 18, 19, 20, 25, 30, 35, 40, 45, 50, 60 70, 80, 90, or 100 times that of non-encapsulated non-cannabinoid therapeutic compounds.

The non-cannabinoid therapeutic compound release profile can be sigmoidal (e.g., having an ‘S’ shape curve, such as a logistic function). The non-cannabinoid therapeutic compound release profile can be non-sigmoidal. The non-cannabinoid therapeutic compound release profile can be linear. The non-cannabinoid therapeutic compound release profile can be non-linear. The non-cannabinoid therapeutic compound release profile can be instant release. The non-cannabinoid therapeutic compound release profile can be non-instant release. The non-cannabinoid therapeutic compound release profile can be delayed release. The non-cannabinoid therapeutic compound release profile can be constant or sustained release. The non-cannabinoid therapeutic compound release profile can be non-constant or non-sustained release.

In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 1 hour of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 2 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 3 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 4 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 5 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 6 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 7 hours of administration to a subject. In some cases, no more than 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a non-cannabinoid therapeutic compound is released from a microcapsule within 8 hours of administration to a subject.

Encapsulation (e.g., microencapsulation) of non-cannabinoid therapeutic compounds can reduce first-pass metabolism of the non-cannabinoid therapeutic compounds in the liver. Non-cannabinoid therapeutic compounds in microencapsulated form can be characterized by a first pass metabolism in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of non-encapsulated non-cannabinoid therapeutic compounds. Non-cannabinoid therapeutic compounds in microencapsulated form can be characterized by an excretion rate from a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of non-encapsulated non-cannabinoid therapeutic compounds.

Non-cannabinoid therapeutic compounds in microencapsulated form can be characterized by a bioavailability in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of non-encapsulated non-cannabinoid therapeutic compounds. Non-cannabinoid therapeutic compounds in microencapsulated form can be characterized by a bioavailability in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%,%, 98%, 99%, or 100%. Non-cannabinoid therapeutic compounds in microencapsulated form can be characterized by a bioactivity in a subject of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0 times that of non-encapsulated non-cannabinoid therapeutic compounds.

The subject composition (e.g., a drug composition in a solid dosage form, such as a tablet or a pill) can comprise a plurality of cannabinoid compounds and a plurality of non-cannabinoid

The composition comprising a non-cannabinoid therapeutic compound and a cannabinoid compound can be characterized by a non-cannabinoid therapeutic compound degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of the cannabinoid compound. Alternatively, the composition can be characterized by a non-cannabinoid therapeutic compound degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than a degradation rate of the cannabinoid compound. Yet in another alternative, degradation rates of a non-cannabinoid therapeutic compound and of a cannabinoid compound can be substantially the same.

The composition comprising a non-cannabinoid therapeutic compound and a cannabinoid compound can be characterized by a non-cannabinoid therapeutic compound plasma half-life in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, 15, 16, 17, 18, 19, 20, 25, 30, 35, 40, 45, 50, 60 70, 80, 90, or 100 times greater than that of a cannabinoid compound. Alternatively, the composition can be characterized by a non-cannabinoid therapeutic compound plasma half-life in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, 15, 16, 17, 18, 19, 20, 25, 30, 35, 40, 45, 50, 60 70, 80, 90, or 100 times less than that of a cannabinoid compound. Yet in another alternative, plasma half-lives of a non-cannabinoid therapeutic compound and of a cannabinoid compound in a subject can be substantially the same.

The composition comprising a non-cannabinoid therapeutic compound and a cannabinoid compound can be characterized by a non-cannabinoid therapeutic compound release rate of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, 15, 16, 17, 18, 19, 20, 25, 30, 35, 40, 45, 50, 60 70, 80, 90, or 100 times greater than that of a cannabinoid compound. Alternatively, the composition can be characterized by a non-cannabinoid therapeutic compound release rate of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5, 6, 7, 8, 9, 10, 11, 12, 13, 14, 15, 16, 17, 18, 19, 20, 25, 30, 35, 40, 45, 50, 60 70, 80, 90, or 100 times less than that of a cannabinoid compound. Yet in another alternative, release rates of a non-cannabinoid therapeutic compound and of a cannabinoid compound from the composition can be substantially the same.

The composition comprising a non-cannabinoid therapeutic compound and a cannabinoid compound can be characterized by a first pass metabolism of the non-cannabinoid therapeutic compound in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than that of a cannabinoid compound. Alternatively, the composition can be characterized by a first pass metabolism of the non-cannabinoid therapeutic compound in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a cannabinoid compound. Yet in another alternative, first pass metabolisms of the non-cannabinoid therapeutic compound and of the cannabinoid compound in a subject can be substantially the same.

The composition comprising a non-cannabinoid therapeutic compound and a cannabinoid compound can be characterized by an excretion rate of the non-cannabinoid therapeutic compound in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than that of a cannabinoid compound. Alternatively, the composition can be characterized by an excretion rate of the non-cannabinoid therapeutic compound in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than that of a cannabinoid compound. Yet in another alternative, excretion rates of the non-cannabinoid therapeutic compound and of the cannabinoid compound in a subject can be substantially the same.

The composition comprising a non-cannabinoid therapeutic compound and a cannabinoid compound can be characterized by a bioavailability of the non-cannabinoid therapeutic compound in a subject of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% greater than that of a cannabinoid compound. Alternatively, the composition can be characterized by a bioavailability of the non-cannabinoid therapeutic compound in a subject of at least 5%, 10%, 15%, 20%, 25%, 30%, 35%, 40%, 45%, 50%, 55% 60%, 65%, 70%, 75%, 80%, 85%, 90%, 95%, 96%, 97%, 98%, or 99% less than that of a cannabinoid compound. Yet in another alternative, bioavailabilities of the non-cannabinoid therapeutic compound and of the cannabinoid compound in a subject can be substantially the same.

In some embodiments, the composition as disclosed herein can comprise a first composition comprising a non-cannabinoid therapeutic compound and a second composition comprising a cannabinoid therapeutic compound. The first composition and the second composition can be administered to a subject sequentially (e.g., one at a time) or simultaneously (e.g., all at once). The first composition and the second composition can be administered to the subject via the same administration route (e.g., both orally) or different administration routes (e.g., one orally and the other intravenously).

In some cases, the first composition and the second composition, as disclosed herein, can be administered to a subject sequentially (e.g., via the same or different administration route(s)). Administration of the first composition to the subject and administration of the second composition to the subject can be separated by at least about 1 minute, 2 minutes, 3 minutes, 4 minutes, 5 minutes, 10 minutes, 15 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 16 hours, 20 hours, 24 hours, or more. Administration of the first composition to the subject and administration of the second composition to the subject can be separated by at most about 24 hours, 20 hours, 16 hours, 12 hours, 11 hours, 10 hours, 9 hours, 8 hours, 7 hours, 6 hours, 5 hours, 4 hours, 3 hours, 2 hours, 1 hour, 50 minutes, 40 minutes, 30 minutes, 20 minutes, 15 minutes, 10 minutes, 5 minutes, 4 minutes, 3 minutes, 2 minutes, 1 minute, or less.

Subjects of the present disclosure can include humans and other animals, such as pets (e.g., dogs, cats, birds, small mammals, snakes) and livestock or farm animals (e.g., cows, pigs, horses, sheep, chickens). Compositions of the present disclosure can be useful for veterinary applications.

Methods and systems of the present disclosure may be used for forming compositions for various uses, such as encapsulating compositions (e.g., therapeutics compositions). Examples of uses of methods of the present disclosure are provided in WO/2016/094810, which is entirely incorporated herein by reference.

### EXAMPLES

### Example 1—Microencapsulation of Cannabinoids

A hemp oil composition is produced, comprising cannabinoids including cannabidiol. Additional essential oils are added to the composition. Alginate (e.g., sodium alginate) and quillaja tree extract are added to the composition. The composition is microencapsulated via a microfluidic nozzle device. Calcium chloride is used to cross-link the microcapsules. The microcapsules are packaged in a suspension, transported, and sold.

### Example 2—Administration of Cannabinoid Composition to a Subject

A cannabinoid composition, such as the microencapsulated cannabinoid composition described in Example 1, is administered to a subject suffering from a cannabinoid deficiency related condition. The level of cannabinoids in the subject increases, and the condition is improved.

### Example 3—Cannabidiol-Rich Hemp and Coconut Oil Product

Hempseed oil is enriched in cannabidiol compounds by addition of hemp stalk and stem extract containing 10% to 40% cannabidiol compounds by weight. The enriched hempseed oil is blended into coconut oil to produce a final composition of about 100 milligrams of cannabidiol compounds in 8 fluid ounces of coconut oil. The coconut oil product is then used to produce consumer products such as moisturizers, lotions, cooking oils, smoothies, spreads, and other food products.

### Example 4—Cannabidiolic Acid-Rich Hemp and Coconut Oil Product

Hempseed oil is enriched in cannabidiolic acid by addition of hemp stalk and stem extract containing 10% to 40% cannabidiolic acid by weight. The enriched hempseed oil is blended into coconut oil to produce a final composition of about 100 milligrams of cannabidiolic acid in 8 fluid ounces of coconut oil. The coconut oil product is then used to produce consumer products such as moisturizers, lotions, cooking oils, smoothies, spreads, and other food products.

### Example 5—Cannabidiol-Rich Hot Chocolate Mix

Hempseed oil is enriched in cannabidiol compounds by addition of hemp stalk and stem extract containing 10% to 40% cannabidiol compounds. Hempseed oil rich in cannabidiol compounds is then combined with cyclodextrin (e.g., certified organic cyclodextrin) to form a dry powder. The hemp oil powder is mixed with powdered cacao, cacao butter mix, sweeteners, and optionally superfood products such as reishi mushroom powder, chaga mushroom powder, maca, or he shou wu. The mixture is packaged and sold as a chocolate beverage mix (e.g., hot chocolate mix).

### Example 6—Production and Packaging of Cannabinoid-Rich Product

A standardized supercritical carbon dioxide extract of hemp stalk and stem is extracted. The extract (e.g., a paste) is blended into hemp seed oil. The blend of hemp extract and hemp oil is prepared with a THC content below 0.3%, and with CBD content of about 10-40% by weight. The hemp extract and hemp oil blend are further blended into coconut oil to provide about 100 mg of CBD per 8 ounce of coconut oil (about 423 milligrams per liter). The coconut oil blend with CBD is packaged (e.g., in ajar) and sold to a consumer.

### Example 7—Administration of Pregnenolone Composition to a Subject

A cannabinoid and pregnenolone composition, such as the microencapsulated cannabinoid composition described in Example 1 further comprising pregnenolone (e.g., 1-50 mg of pregnenolone), is administered to a subject suffering from cannabinoid intoxication or addiction. The subject is protected from CB1 receptor overactivation, and the condition is improved.

### Example 8—Preparation of Microencapsulated Composition

Formulations comprising quillaja extract (e.g., Q Natural), hemp oil, water, and optionally sodium alginate were prepared using a microfluidic fluid processor (e.g., Microfluidizer from Microfluidics/IDEX Corporation). Formulations were prepared as described in Table 11. Test 1 was prepared with 60 g of quillaja extract (e.g., Q Natural), 80 g of hemp oil, and 100 g of water, at an operating pressure of 30,000 psi in the microfluidic fluid processor. Test 2 was prepared with 10 g of quillaja extract (e.g., Q Natural), 15 g of hemp oil, 198 g of water, and 2 g of sodium alginate, at an operating pressure of 30,000 psi in the microfluidic fluid processor.

After processing in the microfluidic fluid processor, particle size distribution was analyzed using a laser diffraction particle size analyzer (e.g., Horiba LA950). Optical microscope images were also taken.

Three passes each of Test 1 and Test 2 were conducted, and the tenth percentile (D10), fiftieth percentile (D50), and ninetieth percentile (D90) particle sizes are reported in Table 11. Particle sizes were also analyzed for an unprocessed solution (Test 1, Pass 0).

TABLE 11
Formulation and size distribution information for microencapsulated compositions.

| | | | | | | Sodium | D10 | D50 | D90 |
|---|---|---|---|---|---|---|---|---|---|
| Test | Pass # | Pressure | Q Natural | Hemp Oil | Water | alginate | (μm) | (μm) | (μm) |
| 1 | 0 | 30,000 | 60 g | 80 g | 100 g | — | 1.9737 | 7.117 | 35.703 |
| | 1 | psi | | | | — | 0.0768 | 0.1296 | 0.2881 |
| | 2 | | | | | — | 0.0697 | 0.1096 | 0.1791 |
| | 3 | | | | | — | 0.0929 | 0.1405 | 0.2202 |
| 2 | 1 | 30,000 | 10 g | 15 g | 198 g | 2 g | 0.1171 | 0.1965 | 2.0719 |
| | 2 | psi | | | | | 0.0688 | 0.1097 | 0.2209 |
| | 3 | | | | | | 0.099 | 0.1583 | 1.3443 |

FIG. 1A shows a 400× magnification micrograph image of an unprocessed quillaja extract, hemp oil, and water composition (Test 1, Pass 0), with a 50 μm scale bar. FIG. 1B shows a 1000× magnification micrograph image of an unprocessed quillaja extract, hemp oil, and water composition (Test 1, Pass 0), with a 50 μm scale bar.

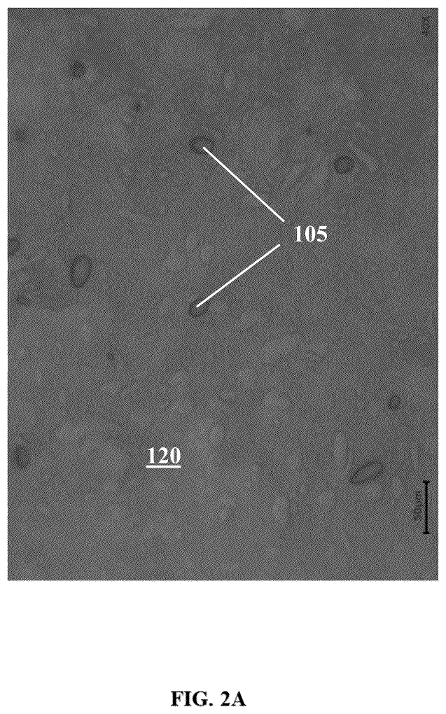

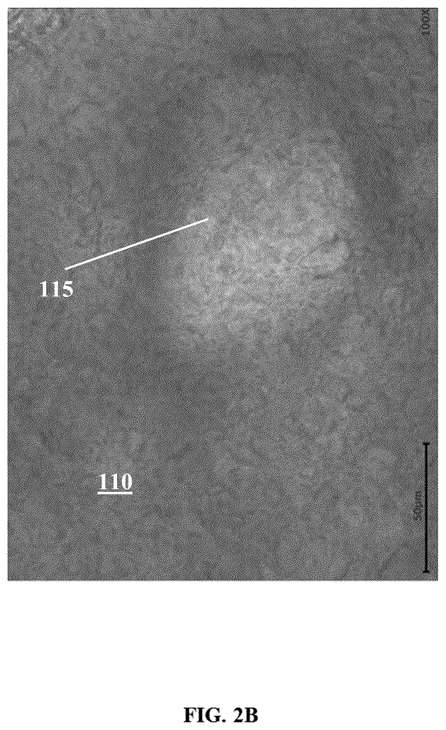

FIG. 2A shows a 400× magnification micrograph image of a quillaja extract, hemp oil, and water composition (Test 1, Pass 1), with a 50 μm scale bar. FIG. 2B shows a 400× magnification micrograph image of a quillaja extract, hemp oil, and water composition (Test 1, Pass 2), with a 50 μm scale bar. FIG. 2C shows a 1000× magnification micrograph image of a quillaja extract, hemp oil, and water composition (Test 1, Pass 2), with a 10 μm scale bar. FIG. 2D shows a 1000× magnification micrograph image of a quillaja extract, hemp oil, and water composition (Test 1, Pass 3), with a 10 μm scale bar.

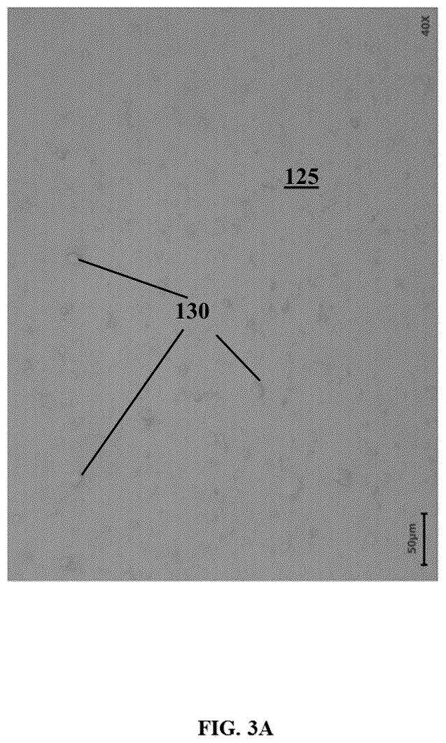

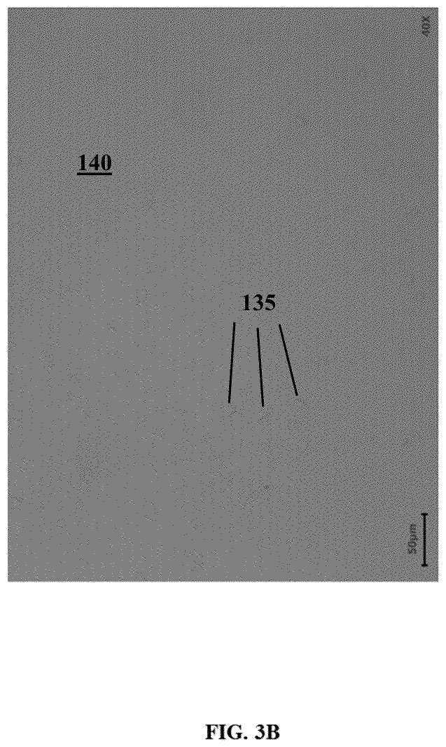

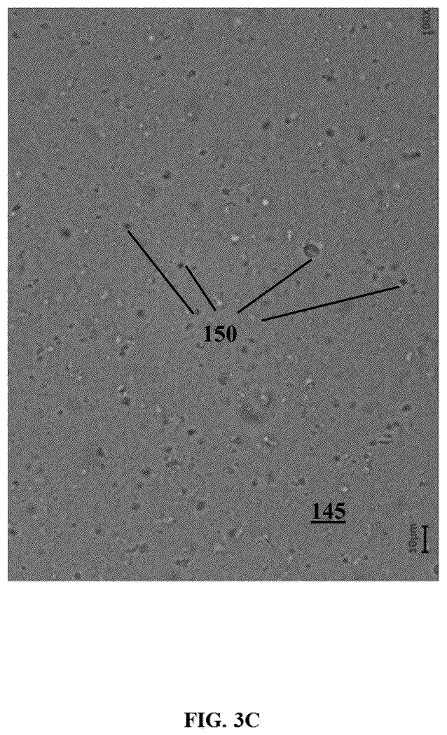

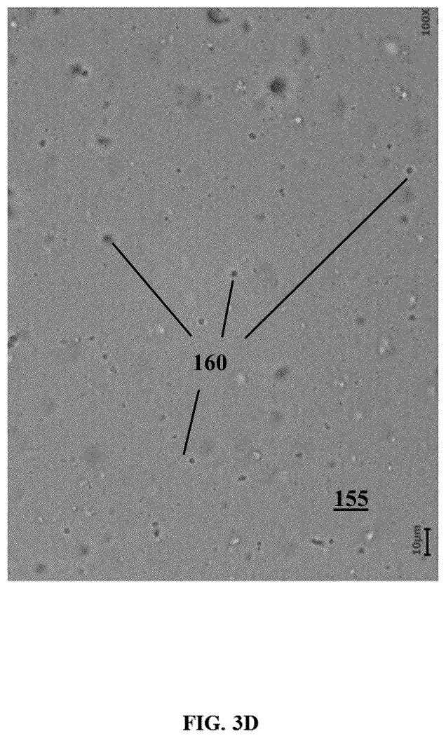

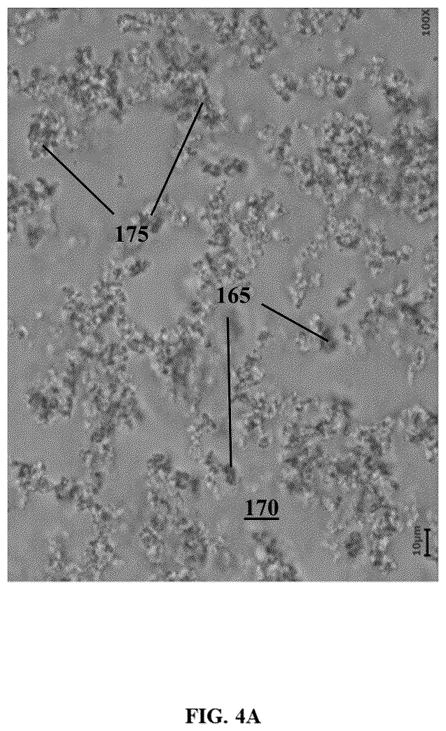

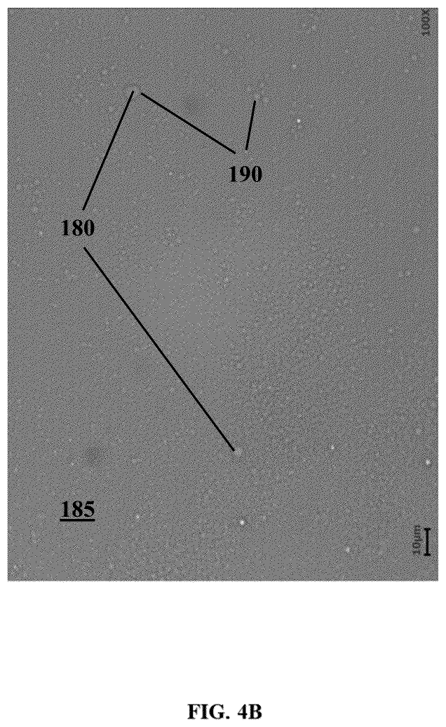

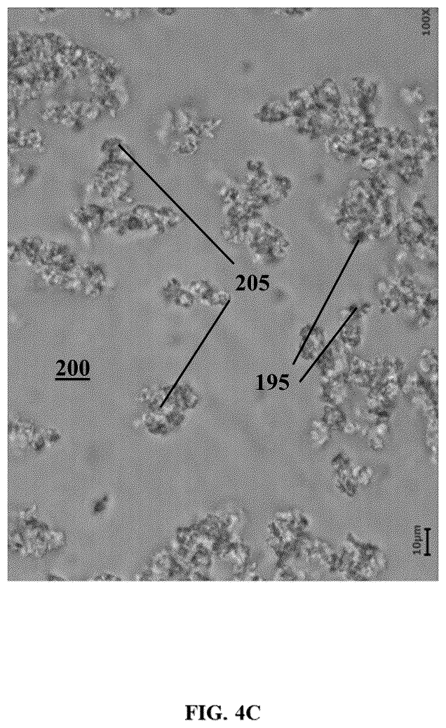

FIG. 3A shows a 1000× magnification micrograph image of a quillaja extract, hemp oil, water, and sodium alginate composition (Test 2, Pass 1), with a 10 μm scale bar. FIG. 3B shows a 1000× magnification micrograph image of a quillaja extract, hemp oil, water, and sodium alginate composition (Test 2, Pass 2), with a 10 μm scale bar. FIG. 3C shows a 1000× magnification micrograph image of a quillaja extract, hemp oil, water, and sodium alginate composition (Test 2, Pass 3), with a 10 μm scale bar.

### Example 9—Administration of Co-Therapeutic Composition to a Subject

A co-therapeutic composition, such as the microencapsulated cannabinoid composition described in Example 1 further comprising a non-cannabinoid therapeutic compound is administered to a subject having a condition or is suspected of having such condition. Subsequent to the administration, the subject is characterized by at least a partial remission of the condition. For example, the subject can have a cancer, and treatment with the co-therapeutic composition as disclosed herein can at least partially treat the cancer (e.g., decrease a size of a solid tumor of the subject). In another example, the subject can have a CNS disease, and treatment with the co-therapeutic composition as disclosed herein can at least partially treat the CNS disease.

### Example 10—Administration of Co-Therapeutic Composition to a Subject

A co-therapeutic composition comprising a non-cannabinoid therapeutic compound and a cannabinoid compound (e.g., a solid composition as illustrated in FIGS. 5 and 6) is administered to a subject having a condition or is suspected of having such condition. Subsequent to the administration, the subject is characterized by at least a partial remission of the condition. For example, the subject can have a cancer, and treatment with the co-therapeutic composition as disclosed herein can at least partially treat the cancer (e.g., decrease a size of a solid tumor of the subject). In another example, the subject can have a CNS disease, as disclosed herein, and treatment with the co-therapeutic composition as disclosed herein can at least partially treat the CNS disease.

### Example 11—Co-Administration of Cannabinoid with Doxorubicin

A co-therapeutic composition comprising a cannabinoid and doxorubicin is administered to a subject undergoing treatment of a cancer via the doxorubicin. Subsequent to the administration, the subject is characterized by at least a partial remission of the condition (e.g., a decrease in a size of a solid tumor of the subject). Subsequent to administration, the subject is further characterized by mitigation of cardiomyopathy via mitochondrial pathways and by activating, binding, or otherwise interacting with endocannabinoid receptors in the heart. Administration of the co-therapeutic composition comprising a cannabinoid and doxorubicin results in an improved patient outcome.

### Example 12—Co-Administration of Cannabinoid with Ketamine

A co-therapeutic composition comprising a cannabinoid and ketamine is administered to a subject who exhibits symptoms of depression. Subsequent to the administration, the subject is characterized by increased antidepressant characteristics and reduced or eliminated psychostimulant effects as compared to an administration of ketamine individually. Subsequent to administration, the subject is characterized by diminished motor disturbance and support of fear extinction in neural networks.

Other examples of the co-administration of a cannabinoid and ketamine are provided in the reference “Co-administration of cannabidiol and ketamine induces antidepressant-like effects devoid of hyperlocomotor side-effects” (Sartim, et al Neuropharmacology 2021, 195, 108679), which is entirely incorporated herein by reference.

### Example 13—Co-Administration of a Cannabinoid with Atorvastatin

A co-therapeutic composition comprising a cannabinoid and atorvastatin is administered to a subject undergoing treatment of high cholesterol via the atorvastatin. Subsequent to the administration, the subject is characterized by at least a partial reduction of cholesterol levels. Subsequent to administration, the subject is characterized by improved (e.g., decreased) cholesterol levels and neuroprotective effects from the administration of the co-therapeutic composition as compared to administration of atorvastatin individually.

### Example 14—Co-Administration of a Cannabinoid with Fluvastatin

A co-therapeutic composition comprising a cannabinoid and fluvastatin is administered to a subject undergoing treatment of high cholesterol via the fluvastatin. Subsequent to the administration, the subject is characterized by at least a partial reduction of cholesterol levels. Subsequent to administration, the subject is characterized by improved (e.g., decreased) cholesterol levels and neuroprotective effects from the administration of the co-therapeutic composition as compared to administration of the fluvastatin individually.

### Example 15—Co-Administration of a Cannabinoid and Dextromethorphan

A co-therapeutic composition comprising a cannabinoid and dextromethorphan is administered to a to a subject undergoing treatment for a cough via the dextromethorphan. Subsequent to the administration, the subject is characterized by at least a partial remission of the cough. Subsequent to administration, subject is characterized by decreased inflammation and enhanced liver protection as compared to administration of the dextromethorphan individually.

### Example 16—Co-Administration of a Cannabinoid with Ibuprofen

A co-therapeutic composition comprising a cannabinoid and ibuprofen is administered to a subject undergoing treatment of pain and inflammation via the ibuprofen. Subsequent to the administration, the subject is characterized by at least a partial remission of the pain and inflammation. Subsequent to administration, subject is characterized by decreased inflammation, decreased pain and enhanced liver protection as compared to administration of the ibuprofen individually.

### Example 17—Co-Administration of a Cannabinoid with Acetaminophen

A co-therapeutic composition comprising a cannabinoid and acetaminophen is administered to a subject undergoing treatment of pain and inflammation via the acetaminophen. Subsequent to the administration, the subject is characterized by at least a partial remission of the pain and inflammation. Subsequent to administration, subject is characterized by decreased inflammation, decreased pain, and enhanced liver protection as compared to administration of the acetaminophen individually.

### Example 18—Co-Administration of a Cannabinoid with Naproxen

A co-therapeutic composition comprising a cannabinoid and naproxen is administered to a subject undergoing treatment of pain and inflammation via the naproxen. Subsequent to the administration, the subject is characterized by at least a partial remission of the pain and inflammation. Subsequent to administration, subject is characterized by decreased inflammation, decreased pain, and enhanced liver protection as compared to administration of the naproxen individually.

### Example 19—Co-Administration of a Cannabinoid and Diazepam

A co-therapeutic composition comprising a cannabinoid and diazepam is administered to a subject undergoing treatment of anxiety and depression via the diazepam. Subsequent to the administration, the subject is characterized by at least a partial remission of the anxiety and depression. Subsequent to administration, subject is characterized by synergistic anxiolytic effects, lower addiction potential, and protection of the subject's liver and brain from the combination of cannabinoid and benzodiazepine as compared to administration of diazepam individually.

### Example 20—Co-Administration of a Cannabinoid and Alprazolam

A co-therapeutic composition comprising a cannabinoid and alprazolam is administered to a subject undergoing treatment of anxiety and depression via the alprazolam. Subsequent to the administration, the subject is characterized by at least a partial remission of the anxiety and depression. Subsequent to administration, subject is characterized by synergistic anxiolytic effects, lower addiction potential, and protection of the subject's liver and brain from the combination of cannabinoid and benzodiazepine as compared to administration of alprazolam individually.

### Example 21—Co-Administration of a Cannabinoid and Clonazepam

A co-therapeutic composition comprising a cannabinoid and clonazepam is administered to a subject undergoing treatment of anxiety and depression via the clonazepam. Subsequent to the administration, the subject is characterized by at least a partial remission of the anxiety and depression. Subsequent to administration, subject is characterized by synergistic anxiolytic effects, lower addiction potential, and protection of the subject's liver and brain from the combination of cannabinoid and benzodiazepine as compared to administration of clonazepam individually.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-72. (canceled)

73. A composition comprising a cannabinoid compound and a non-cannabinoid therapeutic compound,
wherein the non-cannabinoid therapeutic compound is selected from the group consisting of Enbrel, Neulasta, Prolia, XGEVA, Aranesp, Sensipar/Mimpara, Avsola, Otezla, Parsabiv, AndroGel, Creon, Duopa, Huamira, Imbruvica, Niaspan, Orilissa, Skyrizi, Synagis, Trilipix, Advair, Tivicay, Breo/Relvar Ellipta, Ventolin, Lamictal, Avodart, Flovent, Augmentin, Rotarix, Shingrix, Januvia, Janumet, Isentress, Bridion, NuvaRing, Simponi, Zetia/Vytorin, Herceptin, Avastin, Rituxan, Perjeta, Xolair, Actemra, Lucentis, Activase/TNKase, Esbriet, Lantus, Aubagio, Lovenox, Plavix, Myozyme, Toujeo, Dupixent, Fabrazyme, Trulicity, Humalog, Alimta, Cialis, Forteo, Humulin, Taltz, Basaglar, Cymbalta, Xarelto, Eylea, Mirena, Kogenate, Nexavar, Yasmin, Precose, Adalat, Aspirin Cardio, Betaseron, Tagrisso, Symbicort, Brilinta, Farxiga, Nexium, Imfinzi, Pulmicort, Crestor, Lynparza, Faslodex, Eliquis, Opdivo, Sprycel, Orencia SC, Yervoy, Orencia, Revlimid, Baraclude, Empliciti, Nulojix, Stelara, Remicade, Zytiga, Invega Sustenna, Darzalex, Prezista, Imbruvica, Opsumit, Prevnar 13, Lyrica, Ibrance, Lipitor, Xeljanz, Chantix, Sutent, Norvasc, Premarin, Gilenya, Cosentyx, Lucentis, Tasigna, Sandostatin, Gleevec, Afinitor, Galvus, Promacta, Tafinlar, Entyvio, Velcade, Leuprorelin, Azilva, Dexilant, Ninlaro, Genvoya, Truvada, Epclusa, Odefsey, Descovy, Harvoni, Atripla, Biktarvy, Letairis, Ranexa, Copaxone, Bendeka, Methylphenidate Hydrochloride, ProAir HFA, Austedo, QVAR RediHaler, Daptomycin, Metoprolol Succinate, Sildenafil Citrate, Hypochlorous acid, doxorubicin, nano-inducing liposomal doxorubicin, a nano-inducing liposomal taxane, ketamine, a statin, dextromethorphan, a non-steroidal ani-inflammatory drug, and a benzodiazepine.

74. The composition of claim 73, wherein at least one of i) the cannabinoid compound and ii) the non-cannabinoid therapeutic compound is encapsulated.

75. The composition of claim 73, wherein the non-cannabinoid therapeutic compound is the doxorubicin.

76. The composition of claim 73, wherein the non-cannabinoid therapeutic compound is the nano-inducing liposomal doxorubicin.

77. The composition of claim 73, wherein the non-cannabinoid therapeutic compound is the nano-inducing liposomal taxane.

78. The composition of claim 73, wherein the non-cannabinoid therapeutic compound is the ketamine.

79. The composition of claim 73, wherein the non-cannabinoid therapeutic compound is the statin.

80. The composition of claim 79, wherein the statin is atorvastatin, fluvastatin, or lovastatin.

81. The composition of claim 73, wherein the non-cannabinoid therapeutic compound is the velcade.

82. The composition of claim 73, wherein the non-cannabinoid therapeutic compound is the non-steroidal anti-inflammatory drug.

83. The compound of claim 82, wherein the non-steroidal anti-inflammatory drug is ibuprofen, acetaminophen, or naproxen.

84. The composition of claim 73, wherein the non-cannabinoid therapeutic compound is the benzodiazepine.

85. The compound of claim 84, wherein the benzodiazepine is diazepam, alprazolam, clonazepam, or temazepam.

86. The composition of claim 74, wherein the cannabinoid compound is encapsulated.

87. The composition of claim 74, wherein the cannabinoid compound and the non-cannabinoid therapeutic compound are encapsulated.

88. The composition of claim 87, wherein the cannabinoid compound and the non-cannabinoid therapeutic compound are encapsulated in the same microcapsule of a plurality of microcapsules of the composition.

89. The composition of claim 87, wherein the cannabinoid compound and the non-cannabinoid therapeutic compound are encapsulated in different microcapsules of a plurality of microcapsules of the composition.

90. The composition of claim 89, wherein the cannabinoid compound and the non-cannabinoid therapeutic compound are in the same solid dosage form or in different solid dosage form.

91. The composition of claim 73, wherein the composition comprises a first unit dosage form comprising the cannabinoid compound and a second unit dosage form comprising the non-cannabinoid therapeutic compound, wherein the first unit dosage form and the second unit dosage form are for sequential or simultaneous administration to a subject in need thereof.

92. The composition of claim 73, wherein the composition is capable of synergistically yielding a greater degree of therapeutic efficacy in a subject in need thereof, as compared to either one of the cannabinoid compound and the non-cannabinoid therapeutic compound alone.
